(19) United States
(12) Patent Application Publication
(10) Pub. No.: US 2026/0097138 A1
(43) Pub. Date: Apr. 9, 2026

(54) BIOMATERIAL COATED NANOSTRUCTURES FOR PHOTOACOUSTIC IMAGING AND PHOTOTHERMAL THERAPY OF TUMOR LESIONS

(71) Applicants: OSPEDALE SAN RAFFAELE S.R.L., Milano (MI) (IT); ALMA MATER STUDIORUM - UNIVERSITA' DI BOLOGNA, Bologna (BO) (IT)

(72) Inventors: Mauro COMES FRANCHINI, Bologna (BO) (IT); Mirko MATURI, Bologna (BO) (IT); Erica LOCATELLI, Bologna (BO) (IT); Massimo ALFANO, Milano (MI) (IT); Elisa ALCHERA, Milano (MI) (IT); Irene LOCATELLI, Milano (MI) (IT); Flavio CURNIS, Milano (MI) (IT); Angelo CORTI, Milano (MI) (IT)

(73) Assignees: OSPEDALE SAN RAFFAELE S.R.L., Milano (MI) (IT); ALMA MATER STUDIORUM - UNIVERSITA' DI BOLOGNA, Bologna (BO) (IT)

(21) Appl. No.: 19/113,135

(22) PCT Filed: Jul. 27, 2023
(86) PCT No.: PCT/EP2023/070926

§ 371 Date: Mar. 19, 2025

(30) Foreign Application Priority Data
Sep. 28, 2022 (IT) 102022000019992

(51) Int. Cl. A61K 49/22

(52) U.S. Cl. CPC A61K 49/225; A61K 49/221

(57) ABSTRACT

Metal based nanoparticles coated with a polymer functionalized with thiol groups and —NH groups are provided. The polymer can be thiolated chitosan, thiolated and aminated alginic acid, thiolated and aminated hyaluronic acid, or a protein such as albumin or gelatin, or a synthetic thiolated and aminated polymer such as α-thio-ω-amino polyethylene glycols. The groups are linked to a ligand of the integrin family receptors via a heterobifunctional crosslinker bearing functional groups able to bind to amino groups such as N-hydroxysuccinimidyl ester group (NHS ester), an isocyanate group (—NCO), an isothiocyanate group (—NCS), a Sulfo-N-hydroxysuccinimidyl ester group (sulfo-NHS ester), or a carboxylic acid group which is connected by activation with carbodiimide coupling agents; and/or a functional group able to bind thiol groups.

### TECHNICAL FIELD

The present invention is related to a technological platform for diagnostic, therapeutic or theragnostic applications. The invention is related to functionalized agents, preferably metal nanostructures bearing a ligand capable of recognizing a tumor-associated or inflammation-associated component associated to the nanoparticle via a heterobifunctional crosslinker.

In particular the invention is related to metal based nanoparticles
coated with a polymer functionalized with thiol groups and —NH groups, said polymer preferably being selected among thiolated chitosan, thiolated and aminated alginic acid, thiolated and aminated hyaluronic acid, or a protein preferably selected from the group consisting of albumin and gelatin, or a synthetic thiolated and aminated polymer, preferably α-thio-ω-amino polyethylene glycols
wherein said groups are linked to a ligand of the integrin family receptors, preferably a peptide containing an integrin binding motif, and antibody or part of an antibody, a peptidomimetic or an aptamer, via a heterobifunctional crosslinker,
the crosslinker bearing functional groups able to bind to amino groups, preferably selected from the group consisting of N-hydroxysuccinimidyl ester group (NHS ester), an isocyanate group (—NCO), an isothiocyanate group (—NCS), a Sulfo-N-hydroxysuccinimidyl ester group (sulfo-NHS ester), or a carboxylic acid group which is connected by activation with carbodiimide coupling agents;
and/or a functional group able to bind thiol groups, preferably selected from the group consisting of: a maleimide group, a terminal vinyl group or a terminal alkyne group; and/or functional groups able to bind alkynes such as azides; and/or functional groups able to bind azides, such as alkynes.

### BACKGROUND

Early detection and treatment of tumors is fundamental to improve the oncological outcome, survival rate and quality of life of the patients. Bladder cancers (BC) confined to the mucosa and invading the lamina propria are classified as stage Ta and T1, respectively, according to the Tumor, Node, Metastasis (TNM) classification system [1]. Intra-epithelial, high-grade tumors confined to the mucosa are classified as carcinoma in situ (CIS). Approximately 75% of patients with BC present with a disease confined to the mucosa (stage Ta, CIS) or submucosa (stage T1) [2]. All of these tumors can be treated by transurethral resection of the bladder (TURB), eventually in combination with intravesical instillations and are grouped as non-muscle invasive bladder cancer (NMIBC) for therapeutic purposes.

Bladder CIS is characterized by a small number of high-grade neoplastic cells that create a reddish area, indistinguishable from inflammation, and have a flat appearance in the urothelium. It can be missed or misinterpreted as an inflammatory lesion during cystoscopy if not biopsied. The management of patients with bladder CIS still represents a challenge in the onco-urological field [3, 4].

Several imaging methods such as computed tomography urography (CT urography), intravenous urography (IVU), ultrasound (US), multiparametric magnetic resonance imaging (mpMRI) and cystoscopy have been used to attempt the diagnosis of bladder cancer. However, major remaining limitations of diagnostic imaging involve the size of the tumor and the dimensional limit of detectability of any method, with US and CT resulting in very poor detection rates for bladder cancers <5 mm in size [5]. Cystoscopy remains the gold standard diagnostic method for patients with the suspicion of bladder cancer [6]. Indeed, even cystoscopy, including photodynamic diagnosis performed using violet light after intravesical instillation of 5-ALA or hexaminolaevulinic acid, has limited diagnostic utility for CIS. In fact, during cystoscopy and TURB several biopsies from suspicious urothelium should be usually taken to detect and diagnose CIS on surgical tissue specimens [3]. Still, residual high-grade lesions are found in 40% of patients after the first TURB [7]. Due to these technological limitations, patients with bladder CIS experience very high frequencies of relapse following the first diagnosis, thus undergoing frequent and endless follow-up with poorly effective treatments, resulting in a poor quality of life and the highest cost per patient among all cancers [8].

To overcome the limitations of the imaging methods currently used in clinics for bladder CIS, it is felt the need in the art to develop approaches and technologies for a non-invasive early diagnosis of in vivo orthotopic bladder cancer, by exploiting an imaging modality based on the photoacoustic (PA) imaging (PAI) approach. PAI is a hybrid imaging modality that combines the high contrast of optical absorption and the high spatial resolution of US generated by chromophores after irradiation by a non-ionizing pulsed laser. As acoustic waves generally undergo less scattering and tissue attenuation compared to light, PAI can provide higher resolution images than traditional US, and achieve deeper penetration than purely optical imaging systems [9, 10]. PAI also allows for the collection of functional and molecular information in real time by employing non-ionizing radiation to reach clinically relevant imaging depths [11].

Endogenous contrast agents, such as melanin, oxy and deoxy hemoglobin, lipids, collagen and water [12], and pulsed laser light in the near-infrared (NIR) spectral range have been exploited in PAI of melanoma [13], the tumor microenvironment [14], atherosclerotic plaque [15] and injuries [16], respectively. Exogenous contrast agents can also be used to enhance the sensitivity and spectroscopic specificity of PA signals. Targeted contrast agents can also be exploited to extend the range of applications of PAI to molecular imaging [17, 18]. Among the various contrast agents developed so far, gold nanoparticles are of particular interest for their versatility, unique optical and physicochemical properties, relatively inert nature, and successful use in many biomedical applications. In particular, gold nanorods (GNRs) show the highest extinction coefficient in the NIR range and high PA conversion efficiency. Furthermore, tuning the shape of GNRs allows the best wavelength of light stimulation to be selected, thereby enabling the use of these nanoparticles for the needed PAI application [17].

Integrins represent a potential target for human bladder cancer, because they are involved in almost every step of cancer progression from the primary tumor to late stage metastasis development [19]. Among the various integrins that play a role in cancer progression [20], we investigated the expression of the α5β1 integrin, whose overexpression has been reported in high-grade bladder cancer [21, 22] and as a marker of unfavorable prognosis for BC patients [23, 24]. It is therefore felt the need of new technological platform for the diagnosis of non-muscle invasive bladder carcinoma in situ.

### SUMMARY OF THE INVENTION

The purpose of the present invention is to overcome the drawbacks of the known art. In particular, the object of the present invention solves the problems and the limitation presented by the limitations of the imaging methods currently used in clinics for the detection of solid tumors and preferably bladder cancer.

In particular, the object of the present invention are metal based nanoparticles functionalized with a ligand capable of recognizing a tumor-associated or inflammation associated component.

The various embodiments of the invention are presented in the detailed description below and the preferred embodiment are presented in the claims which form an integral part of the present description.

In one embodiment, the invention relates to Metal based nanoparticles
coated with a polymer functionalized with thiol groups and —NH groups, said polymer preferably being selected among thiolated chitosan, thiolated and aminated alginic acid, thiolated and aminated hyaluronic acid, or a protein preferably selected from the group consisting of albumin and gelatin, or a synthetic thiolated and aminated polymer, preferably α-thio-ω-amino polyethylene glycols
wherein said thiol groups and —NH groups are linked to a ligand of the integrin family receptors, preferably a peptide containing an integrin binding motif, and antibody or part of an antibody, a peptidomimetic or an aptamer, via a heterobifunctional crosslinker,
the crosslinker bearing functional groups able to bind to amino groups, preferably selected from the group consisting of N-hydroxysuccinimidyl ester group (NHS ester), an isocyanate group (—NCO), an isothiocyanate group (—NCS), a Sulfo-N-hydroxysuccinimidyl ester group (sulfo-NHS ester), or a carboxylic acid group which is connected by activation with carbodiimide coupling agents;
and/or a functional group able to bind thiol groups, preferably selected from the group consisting of: a maleimide group, a terminal vinyl group or a terminal alkyne group; and/or functional groups able to bind alkynes such as azides; and/or functional groups able to bind azides, such as alkynes.

In a preferred embodiment the metal based nanoparticles gold, silver, or hybrid gold/silver nanoparticles, preferably gold and the nanoparticles have photoacoustic properties and in a further preferred embodiment the nanoparticles are chosen among nanosphere, nanorod, nanostar, nanocage, nanoprism or nanoshell or nanowires, nanoplates, hallow shells, preferably nanorods with a size ranging from 10 to 200 nm in length, and 2 to 50 nm in width, more preferably ranging from 20 to 100 nm in length and from 10 to 25 nm in width and the preferred aspect ratio ranges between 1.2 and 15, more preferably between from 3 and 7.

Preferably the functionalized polymer is thiolated chitosan preferably having an average molecular weight ranging from 0.5 kDa to 1000 kDa, more preferably ranging from 50 kDa to 200 kDa, and an average deacetylation degree ranging from 75% to 100%, more preferably ranging from 85% to 95%.

Preferably, in the above metal based nanoparticles the ligand of the integrin family receptors is a peptide selected from:

[XGisoDGRG] of SEQ ID No: 1
[XisoDGRGG] of SEQ ID No: 2
[XphgisoDGRG] of SEQ ID No: 3
[XGisoDGRphg] of SEQ ID No: 4
[XisoDGRphgG] of SEQ ID No: 5
[XisoDGRGphg] of SEQ ID No: 6
[CphgisoDGRG] peptide of SEQ ID NO: 7
XFETLRGDERILSILRHQNLLKELQD of SEQ ID No: 8
XFETLRGDLRILSILRHQNLLKEL of SEQ ID No: 9
XFETLRGDLRILSILRX₁QNLX₂KELQD of SEQ ID No: 10

wherein “X” is selected from cysteine, lysine or a non-natural amino acid comprising an alkyne or azide group preferably selected from propargylglycine or azidolysine;
X₁ and X₂ are respectively propargyl glycine and azidolysine joined via a triazole bridge.

In a further preferred embodiment the ligand of the integrin family receptors is a peptide comprising the RGD or isoDGR motif, more preferably the peptide is the cyclic head-to-tail [CphgisoDGRG] peptide of SEQ ID NO: 7

Still preferably in the metal based nanoparticles the crosslinker is maleimido-PEG₁₂-NHS ester.

In a preferred embodiment, the invention provides the metal based nanoparticles wherein:
the nanoparticles are gold nanorods
the functionalized polymer is thiolated chitosan
the peptide is [CphgisoDGRG] peptide of SEQ ID No:7.
the crosslinker is maleimido-PEG₁₂-NHS ester.

It is a further object of the invention, a composition comprising the metal based nanoparticles according to previous definitions and at least one of following solvent: water; physiologic solution; Dulbecco's Modified Eagle Medium (DMEM); Dulbecco's phosphate-buffered saline (DPBS), HEPES buffer, TRIS buffer, PIPES buffer each containing metal divalent ions such as Ca2+, Mg2+; preferably said composition comprises at least one or more antitumoral agents preferably chosen among a chemotherapeutic agent, an immunomodulator, an immune cell

It is a further object of the invention a kit comprising single use vials containing the metal based nanoparticles, a solvent, preferably water, or physiologic solution, or Dulbecco's Modified Eagle Medium (DMEM), or a buffer such as Dulbecco's phosphate-buffered saline (DPBS), HEPES buffer, TRIS buffer, PIPES buffer each containing metal divalent ions such as Ca2+, Mg2+, for resuspending the nanoparticles, optionally syringes and instruction for use

Preferably the metal based nanoparticles or compositions or kit are for use in a method of diagnosis and/or treatment in vivo, preferably for use in the in vivo diagnosis and/or treatment of solid tumors, preferably the tumor is selected among urothelial, bladder, gastroesophageal, colorectal, pancreatic, ovarian, lung, cervix, breast and renal cancer, brain tumors and hepatocellular carcinoma.

The invention also provides the above metal based nanoparticles or compositions or kit for use in the photothermal therapy of solid tumors, preferentially bladder cancer. A further object of the invention is a method for ultrasound and photoacoustic imaging ex vivo which comprises at least the following steps:
a) applying the metal based nanoparticles or the composition of the invention to the target tissue to be imaged
b) photoacoustic visualization of the target tissue, and
c) evaluating the visualized target tissue.

Further characteristics and objects of the present invention will become clearer from the following description.

### BRIEF DESCRIPTION OF THE DRAWINGS

FIG. 1—Expression of α5 and β1 integrins in human bladder CIS, and murine non-invasive orthotopic bladder cancer. A) Representative immunohistochemistry photomicrographs of human bladder sections of non-neoplastic tissue and CIS obtained by TURB; one representative image of normal tissue and two representative images of CIS are shown. Tissues were immunostained with the indicated anti-integrins antibodies. Ur; Urothelium, Lp; Lamina propria. Arrows indicate the membrane stain of the α5 integrin.

B) Representative immunohistochemistry photomicrographs of murine orthotopic bladder cancer, 11 days after the intravesical instillation of MB49-Luc cells, and immunostained with the indicated anti-integrins antibodies. One murine bladder out of the 3 analyzed is shown. Upper panels show low magnification (scale bar 500 μm); lower panels show higher magnification of non-neoplastic and neoplastic tissues (scale bar 50 μm).

FIG. 2—Binding of Iso4 peptide to MB49-Luc and 5637 cell lines.

A) Binding of Iso4-Qdot or ARA-Qdot (control) to MB49-Luc and 5637 cells as measured by FACS. A Representative FACS experiment (left) and quantification of Qdot binding (right) are shown. Circles: mean±SEM of duplicates.

B) Binding of Iso4-Qdot or ARA-Qdot to living 5637 cells. Cells were grown in a 96-well plate and incubated with the indicated dose of Qdot (for 2 h, 37° C., 5% CO2), after washing and fixing cell bound fluorescence was acquired using the Cellomics ArrayScan XTI Studio Scan (Thermo Fischer Scientific) system. Magnification 20×; scale bar 10 μm; red, Qdot.

C) Adhesion of MB49-Luc and 5637 cells to solid-phases coated with Iso4-HSA or *HSA (control) and stained with crystal violet. Representative images of wells coated with 30 μg/mL of Iso4-HSA or *HSA (left) and the quantification of cell adhesions (right). Images were acquired with a scanner. Bars, mean±SE, n=4.

FIG. 3—Synthesis and characterization of GNRs@CTAB, GNRs@Chit and GNRs@Chit-Iso4.

A) Steps for the synthesis of GNRs@Chit-Iso4. Chemical modification of chitosan via EDC-coupled amidation of thioglycolic acid with chitosan amino groups; removal of CTAB by the thiolated chitosan that binds GNRs; attachment of the bifunctional PEG linker to the NHS ester terminus to the free amino groups on chitosan; conjugation of Iso4 by exploiting its cysteine residue that is reactive towards the maleimide terminus of the linker.

B) VIS-NIR spectra GNRs@Chit-Iso4 compared to the VIS-NIR spectra of GNRs@CTAB and GNRs@Chit.

C) Shape of GNRs@Chit-Iso4 by TEM analysis (scale bar=50 nm).

D) Distribution of width and length of the nanorods present in the nanosystem GNRs@Chit-Iso4, and the corresponding Gaussian fit (mean±SD and R2 for length 90.2±7.2 nm and 0.86; mean±SD and R2 for width 24.9±2.6 nm and 0.93. n=300 by TEM).

E) Aliquots of GNRs@Chit-Iso4 during the freeze-drying process.

F) VIS-NIR spectra and absorption intensity of GNRs@Chit-Iso4 in the presence of human urine over time.

FIG. 4—GNRs@Chit-Iso4, but not GNRs@Chit, promotes MB49-Luc and 5637 cell adhesion and spreading.

A) Adhesion of MB49-Luc and 5637 cells to microtiterplates coated with various amounts of GNRs@Chit-Iso4 or GNRs@Chit-Cys. Representative images of cell adhesion to 250 μg/ml of gold nanorods (according to dry matter content) and the quantification of cell adhesion are shown (Circles, mean±SE, n=3). The EC50 values reported in each plot are the result of 3-4 independent experiments (mean±SE).

B) Effect of free Iso4 on MB49-Luc and 5637 cell adhesion to microtiterplates coated with 250 μg/ml of GNRs@Chit-Iso4 or GNR@Chit-Cys. MB49-Luc and 5637 cells were mixed with the indicated amount of free Iso4 and left to adhere to microtiter plates coated with GNRs. A representative experiment out of three independent experiments is shown (Circles, mean±SE, n=2).

C) Binding of various amounts of Iso4-HRP or ARA-HRP to microtiter plates coated with or without α5β1 as detected with OPD chromogenic substrate (Circles, mean±SE, n=2).

D) Effect of human urine on the binding of Iso4-HRP to microtiter plates coated with or without α5β1. Iso4-HRP (300 mM) was mixed with the indicated amount of urine, and the mixtures were added to the plates. After washing, bound peroxidase was detected by OPD chromogenic substrate (Bars, mean±SE, n=2).

E) Effect of human urine on MB49-Luc cell adhesion to solid-phase coated with GNRs@Chit-Iso4 or GNRs@Chit-Cys. Cells were suspended in DMEM containing 0.1% BSA (upper panel) or in 25 mM Hepes buffer, pH 7.4, containing 150 mM sodium chloride, 1 mM magnesium chloride, 1 mM manganese chloride and 0.1% BSA (lower panel) and spiked with the indicated amounts of human urine. The mixtures were added to microtiter plates coated with 250 μg/ml of GNRs and left to incubate for 1-2 h at 37° C., 5% CO2. After washing, the adherent cells were fixed and stained with crystal violet. Representative images of cell adhesion to wells coated with 250 μg/ml of gold nanorods and the quantification of cell adhesion are shown (Bars, mean±SE, n=3).

*, P<0.05; **, P<0.01; ***, P<0.001 by two tailed t-test as determined using the GraphPad Prism software.

FIG. 5—In vitro and in vivo PAI of GNRs@Chit-Iso4. A) PAI of agar drop containing GNR@Chit-Iso4 (15 nmol Au) and its associated PA spectrum using 0.6% Intralipid (IL) light-attenuators. The echogenic signal (gray) is generated by the slime in which the agar drop is embedded. The violet ROI delineates the PA signal of GNRs from which the PA spectrum was derived; the PA images were acquired and spectrally unmixed with VevoLab software to separate the contribution of the slime and GNRs (the green signal corresponds to the PA signal of GNRs).

B) Normalized PA spectra of GNRs@Chit-Cys and GNRs@Chit-Iso4 (15 nmoli of Au) embedded in agar drop (one representative experiment of five).

C) 3D distribution of the GNRs@Chit-Iso4 signal in agar drops acquired using the light attenuators made of 0.6% IL.

D) Dose-response plot of the PA signal of GNRs@Chit-Iso4 in agar drop analyzed using light attenuators prepared with the indicated concentration of IL; linear dynamic range from 0 to 3.75 nmol Au, and trend towards plateau from 3.75 to 15 nmol Au (mean±SEM of duplicates are shown).

E) Overlayed PA spectra of GNRs@Chit-Iso4 (3.75 nmol Au) acquired using light attenuators prepared with the indicated concentration of IL.

F) TEM analysis of GNRs@Chit-Iso4 recovered from an agar drop after PA analysis conducted with a light attenuator prepared without IL.

G) Energy fluence at 750, 800 and 850 nm, both in the absence and in the presence of light attenuators containing the indicated amounts of IL (mean±SEM of triplicates).

H) Average energy distribution along the depth, from the Monte Carlo model of the simulated domain reported

I) In vivo PAI of murine bladder after intravesical instillation of 100 μl of vehicles (saline solution) or GNRs@Chit-Iso4 (3 nmol Au), from representative frames taken in the middle of the bladder (one representative experiment of five); the green signal corresponds to the PA signal of GNRs after unmixing the PA signal of melanin, deoxy- and oxy-generated blood and GNRs.

J) PA spectra of the saline solution and of GNRs@Chit-Iso4 (3 nmol Au) in the murine bladder.

K) 3D distribution of the GNRs@Chit-Iso4 (3 nmol Au) PA signal in murine bladder imaged at the indicated time points (one representative experiment of five).

L) 3D distribution of the GNRs@Chit-Iso4 PA signal in the upper and lower half of the murine bladder 30 minutes after instillation, followed by the quantification of the volume occupied by the PA signal (% PA signal of GNRs) in the upper and lower half of the bladder at the different time points (data shown as mean±SEM, each dot representative of one animal). *, **; p value using 2-tailed Mann-Whitney test between upper and lower half of the bladder each time point).

M) PAI of the murine bladder with a well-established tumor on the left side (red asterisk), showing a lack of PA signal after the intravesical instillation of GNRs@Chit-Iso4 (3 nmol Au) and two intravesical washing steps to remove the unbound GNRs; axial diameter of the bladder lumen=3.7 mm. Vol: Volume.

FIG. 6—Early diagnosis of bladder cancer.

A) Axial frame of PAI of an animal with well-established bladder tumors after the intravesical instillation of GNRs@Chit-Iso4 (10 nmol Au), followed by 3 cycles of manual handling of urine, removal of the GNRs and 2 washes with saline solution (one representative experiment of six).

B) Axial frame of PAI of an animal with low-volume bladder tumor after the intravesical instillation of GNRs@Chit-Iso4 (10 nmol Au), followed by 3 cycles of manual handling of urine, removal of the GNRs and 2 washes with saline solution (one representative experiment of six).

C) US scan of the same animal reported in panel C 13 days later the PAI of GNRs.

D) Axial frame of PAI of an animal with US-undetectable bladder tumor after the intravesical instillation of GNRs@Chit-Iso4 (10 nmol Au), followed by 3 cycles of manual handling of urine, removal of the GNRs and 2 washes with saline solution (one representative experiment of six).

E) US scan of the same animal reported in panel E 13 days later the PAI of GNRs.

FIG. 7—Expression of α5 and β1 integrins in Von Brunn's nest and human bladder CIs.

A) One representative immunohistochemistry photomicrographs of two tested human bladder sections of the normal urothelium present in the Von Brunn's nest.

B) Five human bladder CIS with positive stain for α5 integrin at the membrane level, out of six CIS tested. All tissues were from TURB.

FIG. 8—Expression of α5 and β1 integrins in human bladder according to tumor stage in one bladder from radical cystectomy.

Representative immunohistochemistry photomicrographs of human bladder sections of one cancer patient subjected to radical cystectomy with paired non-neoplastic urothelium, NMIBC (pTa and Cis) and MIBC (pT2-pT4) and immunostained with the indicated anti-integrins antibodies. *non-neoplastic urothelium; #; stroma, **tumor tissue. Scale bar: 100 μm.

FIG. 9—Synthesis and characterization of GNRs@CTAB and GNRs@Chit

A) Two liter jacket reactor equipped with mechanical stirrer for the large scale synthesis of GNRs.

B) VIS-NIR spectra of GNRs@CTAB showing the presence of two distinct absorption bands representative of the transversal (left) and longitudinal (right) surface plasmonic resonance.

C) Quantification of gold in GNRs@CTAB by EDX analysis.

D) Shape of GNRs@Chit by TEM analysis (scale bar=50 nm).

E) VIS-NIR spectra of GNRs@Chit.

FIG. 10—TEM and SAED analysis of GNRs@CTAB.

A) Representative TEM image of GNRs@CTAB.

B) and C) selected area electron diffraction of a single GNR, revealing two distinct reflections assigned to the [200] and [222] sets of crystal planes of Au face-centered cubic structure. Two diffraction spots were present: by calculating the corresponding interplanar distances in GNRs electron diffraction, of 2.04 and 1.19 Å have been determined. These correspond to distances between specific planes in gold FCC crystal structure (space group Fm3m), the [200] and [222] sets of planes, respectively.

FIG. 11—1H-NMR spectra of chitosan, thiolated-chitosan and GNRs@Chit at 600 MHz.

A) 1H-NMR (600 MHz, CH3COOH 1% in D2O) of chitosan.

B) 1H-NMR (600 MHz, D2O) of thiolated-chitosan. Full assignment of spectral features was performed by evaluating peak positions and intensities. The peaks corresponding to bound thioglycolic residues (7) are located at 3.2 and 3.5 ppm. By comparison, the obtained spectrum with the ones reported in the literature, the unambiguous assignment of peaks was possible [6]. After conjugation with thioglycolic acid, sharp singlet peaks appear at 3.20 and 3.50 ppm. This has been related to the CH2 residue of the thioglycolic moiety attached to the chitosan amino group, which splits upon partial deprotonation of the thiol group in a neutral aqueous environment.

C) 1H-NMR spectrum of GNRs@Chit (600 MHz, D2O). The obtained spectrum resembles all the features of the chitosan spectrum. The absence of signals coming from CTAB allows to state the success of the ligand exchange reaction. Moreover, it is clearly noticeable that the sharp peak corresponding to the CH2 on thioglycolic moieties does not display splitting due to thiol deprotonation equilibrium. This can be directly connected with the efficient attachment of chitosan on GNRs surface since the thiol group is now bound to gold atoms and protonation equilibrium is not allowed.

FIG. 12—Characterization of GNR@Chit.

A) Thermogram of GNRs@Chit decomposition. The dashed line corresponds to the switch from N2 atmosphere to air. The TGA profile shows a first loss corresponding to −10% weight loss due to the desorption of residual humidity from the lyophilization process. After keeping the sample at 600° C. for 15 min in air, the residual mass, corresponding to the total inorganic content, was as low as 2.1 wt. %.

B) Shear stress-shear rate and C) viscosity-shear rate rotational rheology analysis of GNRs@Chit at 20° C. and 37° C. At low shear rates (<300 Hz) the linearity of the shear stress—shear rate curve suggests Newtonian behaviour of the fluid, confirmed by the fact that viscosity is constant in that range. At higher shear rates viscosity decreases, revealing shear-thinning behaviour with higher applied stresses, and this phenomenon is even more evident at 37° C.

D) Dependence of viscosity with temperature, measured at a constant shear rate of 50 Hz.

FIG. 13—TEM analysis of GNRs@Chit-Iso4.

A) High Angle Annular Dark Field Detector (HAADF) image of GNRs@Chit-Iso4 and the results of Energy-dispersive X-ray spectroscopy (EDX) analysis performed on two isolated GNRs, revealing gold as the only detected atomic component of the GNRs core, with no bromine coming from cytotoxic CTAB was detected. Unlabeled peaks are related to copper and carbon from the sample supporting TEM grid.

B) TEM image of GNRs@Chit-Iso4 and the corresponding selected-area electron diffraction (SAED) pattern confirming the conservation of the crystallinity of the gold core. The reflections corresponding to 1.20 Å, 1.41 Å, 2.03 Å and 2.31 Å have been assigned to the [311], [220], [200] and [111] sets of planes, respectively, by comparing the interplanar distances with tabulated literature data [7].

FIG. 14—Representative HPLC chromatogram of the hydrolyzed products obtained from GNRs@Chit-Iso4 (A) and GNRs@Chit-Cys (B).

Lyophilized GNRs (0.5-1 mM of Au, 0.5-1×10¹¹ NPs/ml) were suspended in water at 5 mg/ml (based on their dry-matter content) and an aliquot was subjected to acidic hydrolysis (20 hours at 110° C., in 6 M hydrochloric acid, 0.1% phenol, 0.1% thioglycolic acid under reduced pressure in an atmosphere nitrogen). The amino acid content in the hydrolyzed product was then quantified by ion exchange chromatography and post-column derivatization with ninhydrin. Arrows indicate the amino acid type (one letter code) clearly detected and quantified over the background (compare with panel B). Amino acid in grey color (three letter code) corresponds to the elution time of a standard amino acid mixture used for calibrating the column. Amino acid in parentheses (three letter code) are below the detection limit. Sarcosine (Sar) was added to the sample as an internal control standard. Note that for d-phg no reference standard exists and in this case the d-phg elutes as methionine; moreover, Cys cannot be quantified using this method.

FIG. 15—Set up of materials and equipment used for in vitro visualization of GNRs by PAI.

A) A representative light attenuator made of 1% agar and IL within dispomold cassettes.

B) Example of 1% agar drops containing GNRs.

C) Instrument set up for PAI of agar drop containing GNRs.

D) US imaging and PA signal of one representative agar drop containing GNRs@Chit-Iso4 (15 nmol) embedded into the slime and acquired using the light attenuator made of 1% agar and 0.6% IL; the echogenic signal (gray) is generated by the slime in which the agar drop is embedded.

E) Laser beam on a photographic paper placed at 8 mm from the laser fibers and exposed to the indicated wavelengths for 5 sec. The length and width of each spot was 13 and 3 mm, respectively.

FIG. 16—Monte Carlo model of light transport.

A) Light fluence distribution within the simulated domain that consists of skin line and the standard tissues. The simulated image shows a very good distribution of light fluence where more fluence is observed at the skin line and then decays along the depth. The color bar shows the fluence distribution where red represents the high fluence and black represents very low fluence.

B) Averaged energy distribution along the depth considering the presence of different IL layers. The light is slightly collimated after penetrating the tissue interface; slightly deeper, both incident and backscattered light are contributing, causing a peak of fluence, which then decays exponentially.

FIG. 17—The binding of GNRs@Chit-Iso4 to neoplastic urothelium is specific.

Three representative axial frames and 3D PAI of GNRs@Chit-Iso4 (1.5 nmol Au, containing 5.6 μg of Iso4) (A), added of 296 μg ARA peptide (B), or 296 μg Iso4 peptide (C) after intravesical instillation followed by manual handling of urine and two washes. One representative mouse of two is shown for each condition, and animals with comparable bioluminescence were used (total flux [p/s] of 7.2×10⁷ in panel A, 1.22×10⁷ in panel B, and 2×10⁷ in panel C).

### DETAILED DESCRIPTION OF THE INVENTION

The aim of the invention is to provide a technological platform for diagnostic, therapeutic or theragnostic applications. While the invention is susceptible to various alternative modifications, some preferred embodiments are described below in detail; said embodiments are example and shouldn't be intended as limiting of the scope of the invention to the specific alternative.

It is therefore object of the present invention the development of functionalized agents, preferably metal nanostructures or nanoparticles bearing a ligand capable of recognizing a tumor-associated or inflammation-associated component.

According to the present invention by nanostructures is meant a chemical substances or materials with particle sizes between 1 to 100 nanometres in at least one dimension. In a preferred embodiment the agents according to the invention are designed to recognize a tumor associated component and to be used in the early diagnosis and/or treatment of solid tumors.

To overcome the obstacles of the available diagnostic tools the inventors have developed a new technological platform based on the use of metal nanostructures coated with a polymer bearing thiol groups and —NH groups coupled to a ligand capable of recognizing a tumor-associated or inflammation-associated component. In a preferred embodiment the nanoparticles according to the invention have photoacoustic properties, more preferably the photoacoustic properties are preferably in the near infrared region I and II.

The ligand according to the invention is coupled to the metal nanostructure via a heterobifunctional crosslinker.

In a preferred embodiment the nanostructures are nanoparticles made of gold, silver, or hybrid gold/silver, the preferred metal being gold.

The nanoparticles according to the invention can be designed having a different shapes; by way of example but non-exclusively they can be designed in the shapes of sphere, rod, star, cage, prism, shell, hallow shell, wire, plates.

In a preferred embodiment the nanoparticles are nanorods, with a size ranging from 10 to 200 nm in length, and 2 to 50 nm in width, more preferably ranging from 20 to 100 nm in length and from 10 to 25 nm in width. In a preferred embodiment the aspect ratio ranges between 1.2 and 15 and preferably between 3 and 7.

In a preferred embodiment the nanoparticles according to the invention are gold nanorods (GNRs) having and aspect ratio of 3.6.

According to the invention the nanoparticles are coated with a polymer bearing thiol groups and —NH groups, including but not limited thiol-bearing polysaccharide as thiolated chitosan (thiol-modified chitosan), thiolated and aminated alginic acid and thiolated and aminated hyaluronic acid, or a protein preferably as of albumin and gelatin, or a synthetic thiolated and aminated polymer, as α-thio-ω-amino polyethyleneglycols. In a preferred embodiment the nanoparticles according to the invention are coated with thioated chitosan, in a more preferred embodiment the nanoparticles are gold nanoparticles coated with thioled chitosan.

The thiolated chitosan according to the invention preferably has an average molecular weight ranging from 0.5 kDa to 1000 kDa, more preferably ranging from 50 kDa to 200 kDa, and an average deacetylation degree ranging from 75% to 100%, more preferably ranging from 85% to 95%. For the introduction of the thiol moieties, chitosan is chemically modified, according to a preferred embodiment with carboxylic acids bearing a free thiol group such as 2-mercaptoacetic acid, 3-mercaptopropionic acid, 4-mercaptobenzoic acid, cysteine, homocysteine, cysteine-containing peptides and proteins and α-thio-ω-amino polyethyleneglycols. The thiol functionalization involves less than 90% of the chitosan free amino groups, more preferably ranging from 75% to 85%.

According to the present invention the coated metal nanoparticles are connected to the aforementioned ligand via a heterobifunctional crosslinker. Said crosslinker bears functional groups able to bind to the amino groups, and/or a functional group able to bind thiol groups.

In a preferred embodiment the functional group able to bind to the amino groups is one of N-hydroxysuccinimidyl ester group (NHS ester), an isocyanate group (—NCO), an isothiocyanate group (—NCS), a Sulfo-N-hydroxysuccinimidyl ester group (sulfo-NHS ester), or a carboxylic acid group which is connected by activation with carbodiimide coupling agents; In a preferred embodiment a functional group able to bind thiol groups, is one of a maleimido group, a terminal vinyl group or a terminal alkyne group; and/or functional groups able to bind alkynes such as azides; and/or functional groups able to bind azides, such as alkynes.

In a preferred embodiment the heterobifunctional linker is maleimido-PEG₁₂-NHS ester.

Therefore a cross-linker is a bifunctional moiety bearing at least two functional groups able to bind to or react with specific groups, wherein the terms bind to or react with have the same meaning. Said at least two functional groups can be the same or different, so that the cress-linker can be homo-functional or heterofunctional. Preferably said at least two functional groups are amino groups and/or functional groups able to bind to or react with thiol groups and/or able to bind or react with alkyne groups and/or able to bind to or react with azide groups. Preferably, the crosslinker according to the invention is a bifunctional moiety bearing functional groups able to bind to amino groups and sulfhydryl groups and/or functional groups able to bind to amino groups and azide/alkyne groups and/or functional groups comprising lipoamide or lipoic acid moiety or sulfhydryl or disulfide containing compounds.

Non limitative examples of the cross-linker according to the invention are reported below.

Succinimidyl-4-(N-maleimidomethyl)cyclohexane-1-carboxylate (SMCC) is a non-cleavable and membrane permeable crosslinker, with the following structure:

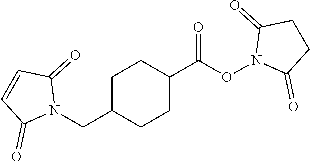 [SMILES: O=C(ON1C(=O)CCC1=O)C1CCC(CN2C(=O)C=CC2=O)CC1]

SMCC contains an amine-reactive N-hydroxysuccinimide (NHS ester) and a sulfhydryl-reactive maleimide group. NHS esters react with primary amines at pH 7-9 to form stable amide bonds. Maleimides react with sulfhydryl groups at pH 6.5-7.5 to form stable thioether bonds.

Sulfo-SMCC (sulfosuccinimidyl 4-(N-maleimidomethyl)cyclohexane-1-carboxylate) corresponds to the compound having the chemical structure indicated below:

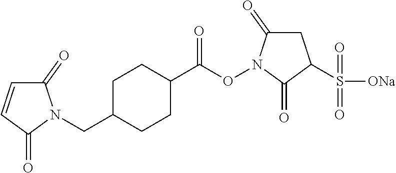

The maleimide groups of SMCC and Sulfo-SMCC (sulfosuccinimidyl 4-(N-maleimidomethyl)cyclohexane-1-carboxylate) and are unusually stable up to pH 7.5 because of the cyclohexane bridge in the spacer arm.

Cross-functional linker of the invention may have the general structure reported below

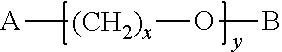

wherein A and B include different reactive groups, x is an integer from 2 to 10 (such as 2, 3 or 4), and y is an integer from 1 to 50, for example, from 2 to 30 such as from 3 to 20 or from 4 to 12.

Non limitative examples of cross-linkers of this structural class are reported below.

Poly(ethylene glycol) (N-hydroxysuccinimide 5-pentanoate) ether N′-(3-maleimidopropionyl)aminoethane (Cas No. 851040-94-3; MAL-PEG-NHS ester):

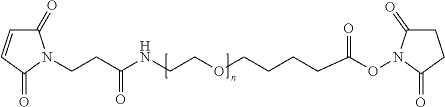

Mal-amido-PEG-TFP ester indicates a PEG linker containing a maleimide and TFP ester end group. Maleimide groups are reactive with thiols between pH 6.5 and 7.5. The TFP ester can react with primary amine groups and is also less susceptible to undergo hydrolysis compared to NHS ester. The hydrophilic PEG chains increase the water solubility of a compound in aqueous media. Longer PEG chains have improved water solubility relative to shorter PEG chains. The PEG linker has a variable number of glycol units, such as the MAL-dPEG®8-TFP ester, MAL-dPEG®4-TFP ester

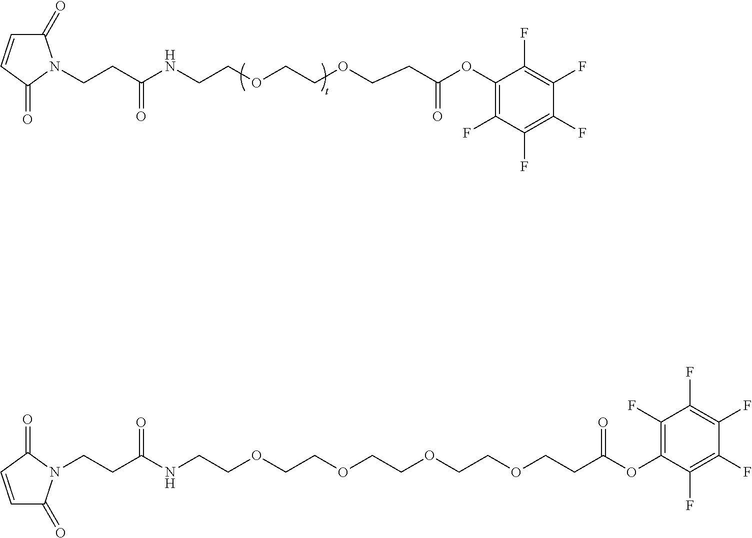

Propargyl-PEG-NHS ester is an amine reactive reagent that can be used for derivatizing peptides, antibodies, amine coated surfaces etc. The alkyne group reacts with azide-bearing compounds or biomolecules in copper catalyzed Click Chemistry reactions. It comprises for example propargyl-PEG1-NHS ester with one glycol unit and propargyl-PEG4-NHS ester which is a 4-unit amine reactive PEG linker

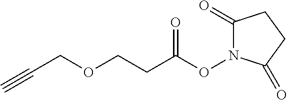 [SMILES: C#CCOCCC(=O)ON1C(=O)CCC1=O]

Azido-PEG-TFP ester is a click reagent containing an azide group and a TFP moiety. The azide group enables Click Chemistry. The TFP ester is can be used to label the primary amines (—NH2) of proteins, amine-modified oligonucleotides, and other amine-containing molecules. Chemical structure of representative compound (2,3,5,6-Tetrafluorophenyl 3-[2-[2-[2-(2-azidoethoxy)ethoxy]ethoxy]ethoxy]propanoic acid) is depicted below:

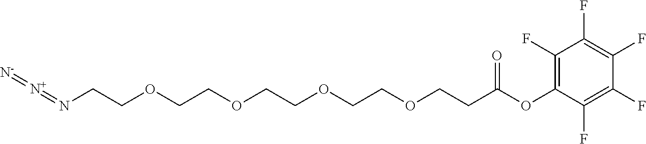 [SMILES: [N-]=[N+]=NCCOCCOCCOCCOCCC(=O)Oc1c(F)c(F)c(F)c(F)c1F]

Similar PFP derivative (perfluorophenyl 1-azido-3,6,9,12-tetraoxapentadecan-15-oate; N3-PEG4-PFP) has the structure below:

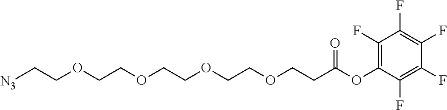 [SMILES: [N-]=[N+]=NCCOCCOCCOCCOCCC(=O)Oc1c(F)c(F)c(F)c(F)c1F]

Azido-PEG-NHS ester is a PEG reagent which contain an azide group and an NHS ester. A non limitative example is the 2,5-Dioxo-1-pyrrolidinyl 3-[(23-azido-3,6,9,12,15,18,21-heptaoxatricos-1-yl)oxy]propanoate:

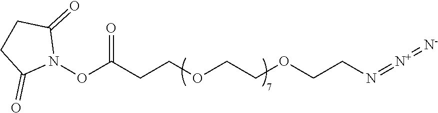

### Thiol PEG

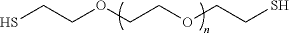

### Thiol-PEG-acid

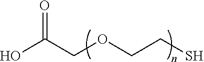

### Thiol-PEG-amine

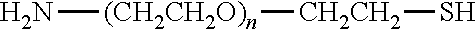

### Thiol-PEG-azide

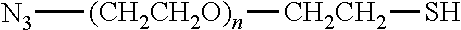

### Propargyl-PEG-MAL

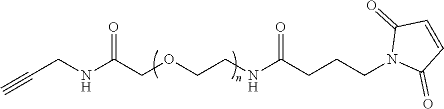

### Azido-PEG-MAL

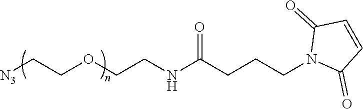

In the above compounds the polyethylene glycol chain (PEG) has a molecular weight comprise between 0.05-40 KDa, preferably lipoamide/lipoic acid-PEG-MAL and the PEG has a molecular weight of 5 KDa.

According to the present invention the nanostructures coated with a polymer bearing thiol groups and —NH groups are coupled via the aforementioned heterobifunctional crosslinker to a ligand capable of recognizing a tumor-associated or inflammation-associated component. According to the invention it is preferred a ligand of the integrin family receptors, a protein (more preferably and antibody or part of an antibody), a peptide, a peptidomimetic or an aptamer. The integrins are chosen as they are key regulators of cell structure and behaviour, affecting cell morphology, proliferation, survival and differentiation.

In a preferred embodiment the integrin family receptor is selected from the group consisting αvβ1, α8β1, α5β1, αvβ3, αvβ5, αvβ6, αvβ8, α3β1, α6β1, α7β1, α6β4, α1β1, α2β1, α10β1, α11β1, α4β1 and α9β1, α5β1 being preferred.

According to the invention the ligand can be a peptide comprising an integrin binding motif as RDG (Arg-Gly-Asp) or isoDGR; in a preferred embodiment the peptide consists of a cyclic isoDGR peptide, selected from the group of

[XGisoDGRG], of SEQ ID No: 1
[XisoDGRGG], of SEQ ID No: 2
[XphgisoDGRG], of SEQ ID No: 3
[XGisoDGRphg], of SEQ ID No: 4
[XisoDGRphgG], of SEQ ID No: 5
[XisoDGRGphg] of SEQ ID No: 6

wherein “X” is preferably a cysteine, a lysine or any alkyne- or -azide functionalized amino acid such as propargylglycine or azidolysine

In another preferred embodiment the ligand is a linear peptide selected from the group of

XFETLRGDERILSILRHQNLLKELQD, of SEQ ID No: 8
XFETLRGDLRILSILRHQNLLKEL, of SEQ ID No: 9
XFETLRGDLRILSILRX₁QNLX₂KELQD, of SEQ ID No: 10

wherein “X” is preferably a cysteine, a lysine or any alkyne- or -azide functionalized amino acid such as propargylglycine or azidolysine, whereas X₁ and X₂ in form a triazole bridge via a propargylglycine (X₁) and azidolysine (X₂).

In a preferred embodiment the ligand is [CphgisoDGRG] of SEQ ID No:7 and structure below:

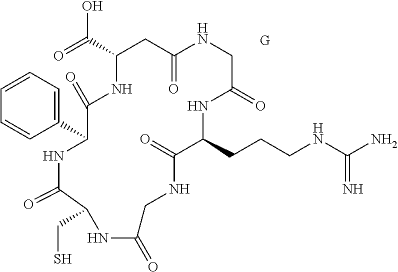 [SMILES: N=C(N)NCCC[C@@H]1NC(=O)CNC(=O)C[C@@H](C(=O)O)NC(=O)[C@H](c2ccccc2)NC(=O)[C@H](CS)NC(=O)CNC1=O]

The compound can exists as a mixture of isomers corresponding to a cyclic head-to-tail peptide with D-phenylglycine (D-phg) and L-phenylglycine (L-Phg).

As used herein “phg” indicates D-phenylglycine (D-phg) and “Phg” indicates L-phenylglycine (L-Phg). The ratio of D-phenylglycine (D-phg) and L-phenylglycine (L-Phg) can be comprised between 50:50 and 99:1, 60:40 and 90:10, 65:35 and 80:20. In a representative example the compound comprises about 70% of D-phenylglycine (D-phg) and about 30% of L-phenylglycine (L-Phg).

As used herein, “isoD” indicates isoaspartic acid (isoaspartate, isoaspartyl, β-aspartate), which is an aspartic acid residue isomeric to the typical α peptide linkage. It is a β-amino acid, with the side chain carboxyl moved to the backbone.

Head-to-tail cyclized peptides are peptide with a cyclic structure. Head-to-tail backbone (homodetic) cyclization represents a peptide modification that imparts rigidified structure, biorelevant turn conformations, increased proteolytic stability, and improved membrane permeability.

Therefore are object of the present invention metal based nanoparticles, preferably gold nanoparticles, more preferably gold nanorods, coated with a polymer functionalized with thiol groups and —NH groups, said polymer preferably being selected among thiolated chitosan, thiolated and aminated alginic acid, thiolated and aminated hyaluronic acid, or a protein preferably selected from the group consisting of albumin and gelatin, or a synthetic thiolated and aminated polymer, preferably α-thio-ω-amino polyethylene glycols, wherein said groups are linked to a ligand of the integrin family receptors, preferably a peptide containing an integrin binding motif, and antibody or part of an antibody, a peptidomimetic or an aptamer, via a heterobifunctional crosslinker, the crosslinker bearing functional groups able to bind to amino groups, preferably selected from the group consisting of N-hydroxysuccinimidyl ester group (NHS ester), an isocyanate group (—NCO), an isothiocyanate group (—NCS), a Sulfo-N-hydroxysuccinimidyl ester group (sulfo-NHS ester), or a carboxylic acid group which is connected by activation with carbodiimide coupling agents; and/or a functional group able to bind thiol groups, preferably selected from the group consisting of: a maleimide group, a terminal vinyl group or a terminal alkyne group; and/or functional groups able to bind alkynes such as azides; and/or functional groups able to bind azides, such as alkynes.

According to the present invention the preferred agents are gold nanorods, coated with thiolated chitosan wherein the heterobifunctional linker is maleimido-PEG₁₂-NHS ester and the ligand is the [CphgisoDGRG] peptide.

Any of the agents of the described embodiment can be prepared in solution or can be prepared in a lyophilized form. With the term “lyophilized” is meant also dried, freeze-dried or precipitated by means of water-miscible organic solvents such as acetone

It also object of the present invention a composition comprising the described theragnostic agents; the composition according to the invention comprises at least one of water, physiologic solution/saline, Dulbecco's Modified Eagle Medium (DMEM), Dulbecco's phosphate-buffered saline (DPBS), HEPES buffer, TRIS buffer, PIPES buffer each containing metal divalent ions such as Ca²⁺, Mg²⁺, a pharmaceutical acceptable excipient, a pH regulator.

In particular, for use in treatment, the composition can be prepared comprising, in addition to the above described components, one or more medicament, in particular at least one of a chemotherapeutic agent, a immunomodulator, an immune cell.

In a preferred embodiment chemotherapeutic agent is selected from the group of: mitomycin-C, Bacillus Calmette Guerin (BCG), doxorubicin, melphalan, gemcitabine, taxol, cisplatin, vincristine, or vinorelbine; more preferably the immunomodulator is an anticancer vaccine and/or an immune check point blocker, such as anti-PD1 or anti-PDL1 or anti-CTLA4 antibodies, and more preferably the immune cell is a lymphocyte or a genetically modified T-lymphocyte, such as CAR-T cells, or TCR redirected T-cells or NK cells.

In a preferred embodiment the invention encompasses a composition comprising gold nanoparticles, preferably nanorods according to the invention, coated with thiolated chitosan, wherein the heterobifunctional linker is maleimido-PEG₁₂-NHS ester and the ligand is the [CphgisoDGRG] peptide and Dulbecco's Modified Eagle Medium (DMEM) or Dulbecco's phosphate-buffered saline (DPBS) with calcium and magnesium.

It is also object of the present invention a kit comprising single use vials containing the agent as described above, a solvent, preferably physiologic solution/saline, Dulbecco's Modified Eagle Medium (DMEM), Dulbecco's phosphate-buffered saline (DPBS), HEPES buffer, TRIS buffer, PIPES buffer each containing metal divalent ions such as Ca²⁺, Mg²⁺, for resuspending the nanoparticles, optionally syringes and instruction for use.

It is also object of the present invention the application of the described agents, compositions and kits in the medical and diagnostic field.

In particular the nanoparticles object of the present invention with photoacoustic properties are developed for use in the diagnosis of solid tumors or inflammation and can be used in particular for the early detection of small cancer lesion, in particular urothelial, bladder gastroesophageal, colorectal, pancreatic, ovarian, lung, cervix, breast and renal cancer, brain tumors and hepatocellular carcinoma. The nanoparticles, as disclosed by the present invention, are particularly suitable to detect urothelial and bladder cancer and actinic cystitis chronic by photoacoustic imaging.

In a preferred embodiment nanoparticles according to the present inventions can be used in the early diagnosis of bladder lesions, in particular of bladder cancer. Are therefore object of the present invention the disclosed nanoparticles for use in a method of in vivo diagnosis of bladder cancer in particular of small and flat urothelial lesions of high-grade bladder carcinoma in situ (CIS).

It is in fact possible to proceed with intravesical instillation of urine-stable nanoparticles designed according to the invention, having photoacoustic properties, handling urine containing the said nanoparticles in the bladder to avoid sedimentation and then use a multimodal imaging of the targeted lesions with PAI.

In a preferred embodiment the targeted area can be subject to thermal ablation; the delivery of the continuous light will irradiate the targeted area with the bound nanoparticles. Assisted photothermal therapy is generated by the excitation of particles at a chosen wavelength.

It is therefore object of the present invention the application of the described agents, composition and kits in the medical and diagnostic field, in particular for theragnostic application.

In a preferred embodiment nanoparticles or compositions according to the present inventions can be used in combination with a medicament, in particular chemotherapeutic agent, a immunomodulator, an immune cell in a method of combination therapy wherein the administration of the nanoparticles and of the medicament can be simultaneous, contemporaneous or sequential.

In a preferred embodiment chemotherapeutic agent is selected from the group of: mitomycin-C, Bacillus Calmette Guerin (BCG), doxorubicin, melphalan, gemcitabine, taxol, cisplatin, vincristine, or vinorelbine; more preferably the immunomodulator is an anticancer vaccine and/or an immune check point blocker, such as anti-PD1 or anti-PDL1 or anti-CTLA4 antibodies, and more preferably the immune cell is a lymphocyte or a genetically modified T-lymphocyte, such as CAR-T cells, or TCR redirected T-cells or NK cells.

In order to demonstrate the effectiveness of the targeted agents according to the invention and in particular of nanoparticles object of the present invention the inventors proceeded to design Gold nanorods (GNRs) that have been chemically engineered with chitosan (Chit) and the peptide Iso4 (head-to-tail cyclized c(CphgisoDGRG) peptide, selective for the α5β1 integrin (ki=15 nM) [25]) to enable tumor targeting; said particles are developed for use in a method of in vivo diagnosis and treatment of bladder cancer based on the intravesical instillation of said urine-stable targeted GNRs (called GNRs@Chit-Iso4); moving the suspension of GNRs@Chit-Iso4, to prevent nanoparticle sedimentation in the bladder; and, the multimodal imaging of cancer lesions with PAI.

The inventors developed a combination of strategies that allow for the detection of this tumor with an unprecedented sensibility. The results show that the combination of PAI with moving of intravesical instilled GNRs@Chit-Iso4 in an orthotopic model of bladder cancer can reveal the presence of lesions undetectable with US imaging and bioluminescence. The technological platform, according to the invention, could detect neoplastic lesions smaller than a half millimeter, with a sensitivity that far exceeds that of the US and CT urography for bladder carcinoma [5]. The inventors were able to realize GNRs that can be used to detect orthotopic murine bladder cancer lesions <0.5 mm, undetectable by US imaging and bioluminescence. The inventors were able to detect up to 250 m neoplastic lesion in the upper part of the bladder of experimental mice. According to a preferred embodiment the targeted nanoparticles of the invention are gold nanorods designed with a diameter of 10 nm and aspect ratio of 3.6 in order to have a peak light absorption at 808 nm, to leverage the optical window that allows for deeper tissue penetration [40, 41] and to overcome the different endogenous contrast molecules present in tissues [18]. It was investigated and established the PA dynamic range of the above nanoparticles and identified the maximum fluence and energy of the pulsed laser light to obtain diagnostic imaging using targeted nanoparticles avoiding reshaping of the nanostructure.

The urinary bladder environment in particular offers the possibility to exploit the intravesical instillation of the targeted nanoparticles, which is characterized by pro and cons compared to systemic delivery. Intravesical instillation allows for the avoidance of off target effects and off-target accumulations, such as the accumulation of gold in the liver, spleen, kidney, testis and brain, as has been observed in cases of systemic instillation [42, 43]. On the other hand, one of the problem to be solved is that intravesical delivery of the treatment i) must content with urine, which contains a broad variety of byproducts from the metabolism of endogenous and exogenous substances [37], bacteria [38, 44], bacteria-derived mucus and floating urothelial cells, ii) is characterized by temporary retention, and iii) cannot exploit the enhanced permeability of the tumor vasculature and retention effects of the neoplastic vasculature to accumulate the intravenously injected target nanoparticles in the neoplastic environment.

The first step in developing the nanoparticle was therefore the identification of a target expressed only in the tumor cells and not expressed in the non-neoplastic bladder epithelium to direct them only on the tumoral tissue.

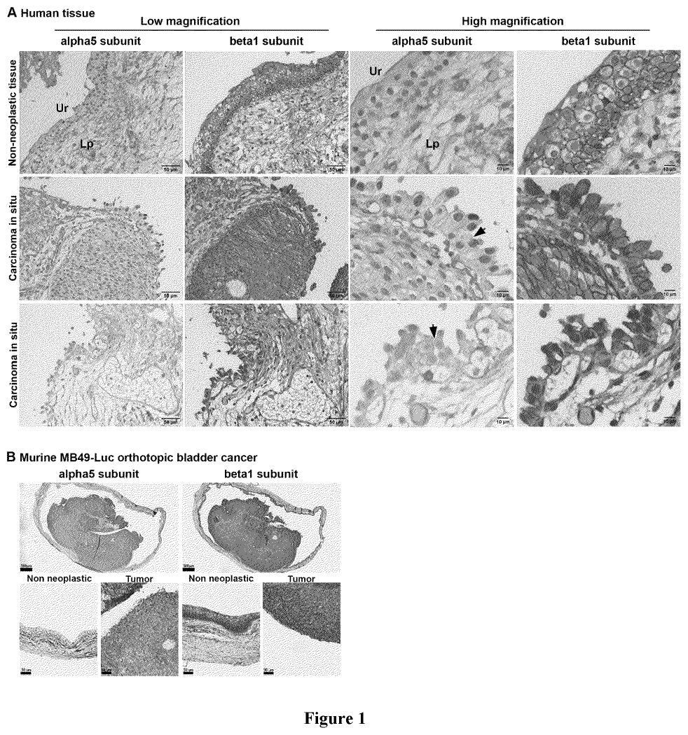

Integrins represent a potential neoplastic target for human bladder cancers, as they are implicated in almost every step of cancer progression from the primary tumor to late stage metastasis development. Among the various integrins with a role in cancer progression, the inventors investigated the cellular receptor for fibronectin, whose expression levels increase in the tumor stroma in association with the tumor stage, for the expression of the α5β1 integrin 7. The α5β1 integrin has been reported to be both a marker of unfavorable prognosis for bladder cancer patients and also overexpressed by high-grade invasive bladder cancer. To understand if α5β1 integrin could be a potential receptor for targeting non-infiltrating tumors its expression in non-neoplastic and neoplastic bladder tissues was evaluated through immunohistochemical analysis of tissue sections obtained from TURB specimens with a histological diagnosis of CIS. The α5 subunit was not expressed by the non-neoplastic urothelial cells, while membrane staining was observed in the CIS; stromal cells in the lamina propria of non-neoplastic and neoplastic bladder tissues showed similar expression (FIG. 1A). The β1 subunit was strongly expressed by non-neoplastic urothelial cells and stromal cells, as well as by the CIS (FIG. 1A). Lack of α5 subunit expression in the normal urothelium was also observed in the Von Brunn's Nests and the expression of the α5 subunit in CIS was confirmed in five out of six TURBs (FIG. 7) while the β1 subunit was always expressed by the normal urothelium and stromal cells. In one out of two radical cystectomies we confirmed the expression of the α5 subunit in the CIS and a zonal expression in non-infiltrating papillary, while little or no expression was observed in the infiltrating tumor pT1, pT2 and pT4 (FIG. 8). Therefore, α5β1 might represent a potential receptor for targeting non-infiltrating tumors such as the non-infiltrating papillae and CIS.

Next, inventors checked the expression of the α5β1 integrin on two non-tumoral human primary urothelial cells (PCS-420-010 and HBLAK) and on four bladder cancer cell lines (RT4, RT112, 5637 and HT1376) derived from human tumors of different stage and grade [34] through flow cytometry analysis using a different set of anti-integrin antibodies. The results represented in Table 1 below showed that human bladder cancer cell lines express more α5 and β1 than human primary urothelial cells.

TABLE 1

| | | Primary Urothelial Cells | | | | Bladder carcinoma cell lines | | | | | | | |
|---|---|---|---|---|---|---|---|---|---|---|---|---|---|
| | | PCS-420- | | | | RT4 ^{b} | | RT112 ^{a} | | 5637 ^{a} | | HT1376 ^{a} | |
| | mAb | 010 ^{a} | | HBLAK ^{b} | | (T1, G1-2) ^{c} | | (Ta, G2) ^{c} | | (G2) ^{c} | | (>T2; G3) ^{c} | |
| Antigen | clone | n ^{d} | Fold ^{e} | n | Fold | n | Fold | n | Fold | n | Fold | n | Fold |
| α5 | P1D6 | 1 | 1.5 | 1 | 2.1 | 2 | 6.0 ± 3.8 | 3 | 5.8 ± 2.2 | 2 | 1.9 ± 0.1 | 3 | 4.1 ± 0.8 |
| β1 | P5D2 | 1 | 1.2 | 1 | 0.8 | 2 | 18.6 ± 4.8 | 3 | 38.7 ± 19.9 | 3 | 40.5 ± 16.6 | 3 | 11.9 ± 2.9 |

^{a} Derived from a female donor.
^{b} Derived from a male donor.
^{c} Stage and tumor grade of cancer cells.
^{d} n, number of independent experiments each in duplicate.
^{e} Fold, ratio of the median fluorescence intensity of a given anti-integrin antibody over the median fluorescence intensity of an isotype control matched antibody, mean ± SEM.

Taken together these results suggest that the expression of α5β1 by human bladder CIS may represent a potential target for the development of new tumor targeted diagnostic tools based on α5β1 targeting.

The inventors also investigated whether MB49-Luc tumor bearing-mice, a widely used syngeneic model in orthotopic bladder cancer [35], could recapitulate the α5β1 expression pattern observed in the human bladder CIS. The results showed that the α5 subunit was expressed by MB49-Luc tumor cells, but not by the non-neoplastic adjacent epithelial cells. In contrast, stromal cells of both neoplastic and non-neoplastic tissues showed a similar level of expression (FIG. 1B). The β1 subunit was expressed by the basal urothelial cells in the non-neoplastic and neoplastic tissue (FIG. 1B). Accordingly, as represented in table 2 below, flow cytometry analysis of MB49-Luc bladder cancer cell stained with the indicated rabbit anti-integrin antibodies followed by a goat anti-hamster Alexa Fluor 488-labeled secondary antibodies confirmed that MB49-Luc cells express the α5β1 integrin.

TABLE 2

MB49-Luc

| Antigen | mAb clone | n ^{a} | Fold ^{b} |
|---|---|---|---|
| α5 | HMα5-1 | 2 | 3.79 ± 0.18 |
| β1 | HMβ1-1 | 2 | 8.02 ± 1.04 |

^{a} n, number of independent experiments each in duplicate.
^{b} Fold, ratio of the mean fluorescence intensity of a given anti-integrin antibody over the mean fluorescence of an isotype control matched antibody, mean ± SEM.

Therefore, mice bearing the MB49-Luc derived orthotopic tumor could be used as a preclinical model for the development of new tumor targeted strategies based on α5β1 targeting. These experiments confirmed that integrin α5β1 is indeed a good target as it is being expressed by the human bladder CIS and human bladder cancer cell lines, as well as expressed by the murine MB49-Luciferase (MB49-Luc) cell line and the MB49-Luc derived orthotopic syngeneic murine bladder cancer [51], but not by both the non-neoplastic human and murine urothelium.

The inventors decided to investigate the use of the Iso4 peptide that was previously reported to selectively recognize the α5β1 integrin [25]. There was the need to assess whether Iso4 can recognize α5β1-positive bladder cancer cells, and also to investigate and demonstrate if the peptide Iso4 could maintains its functional properties (i.e. α5β1 integrin recognition) even after coupling to GNRs@Chit and above all in the presence of urine, and that upon coupling to GNRs the PA spectra of the nanoparticles was not modified.

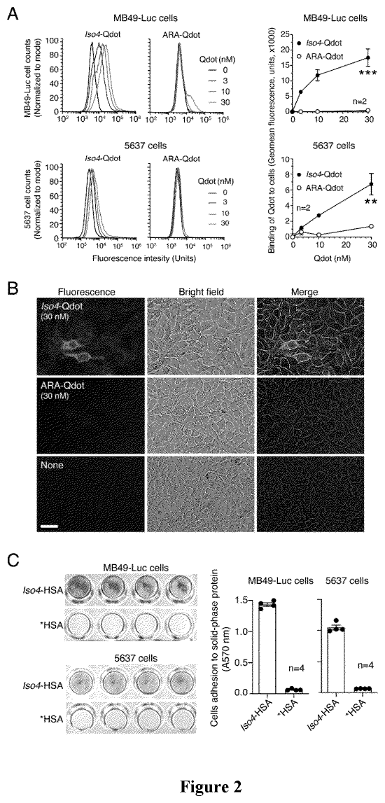

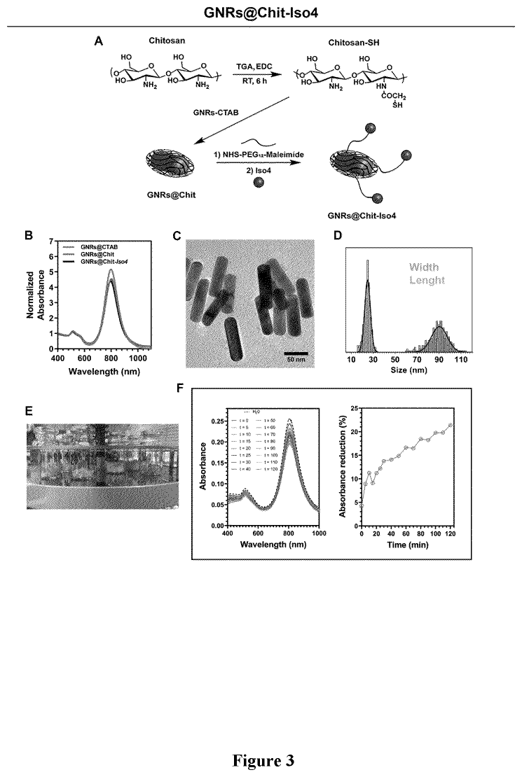

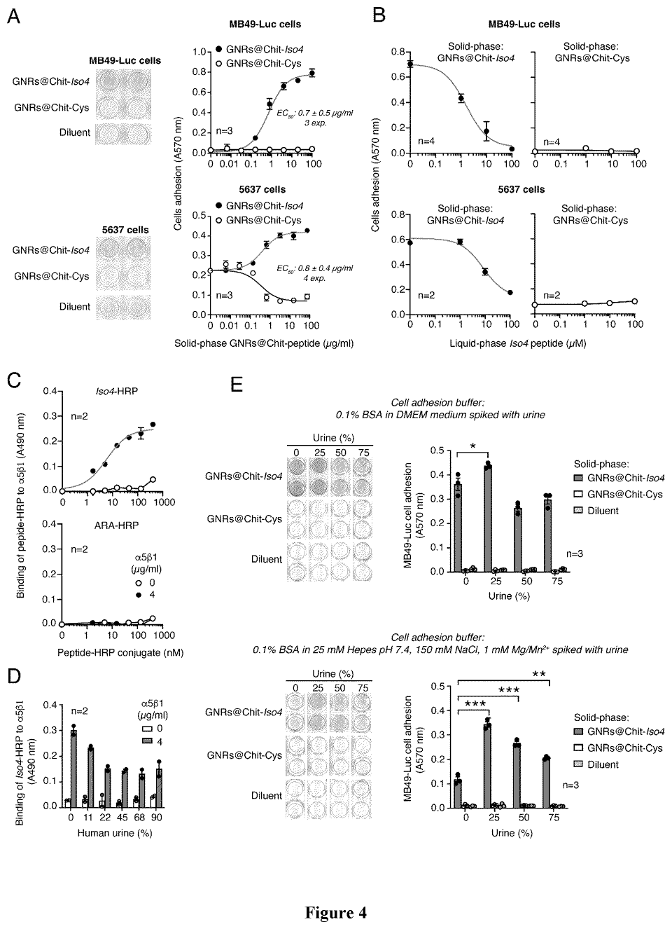

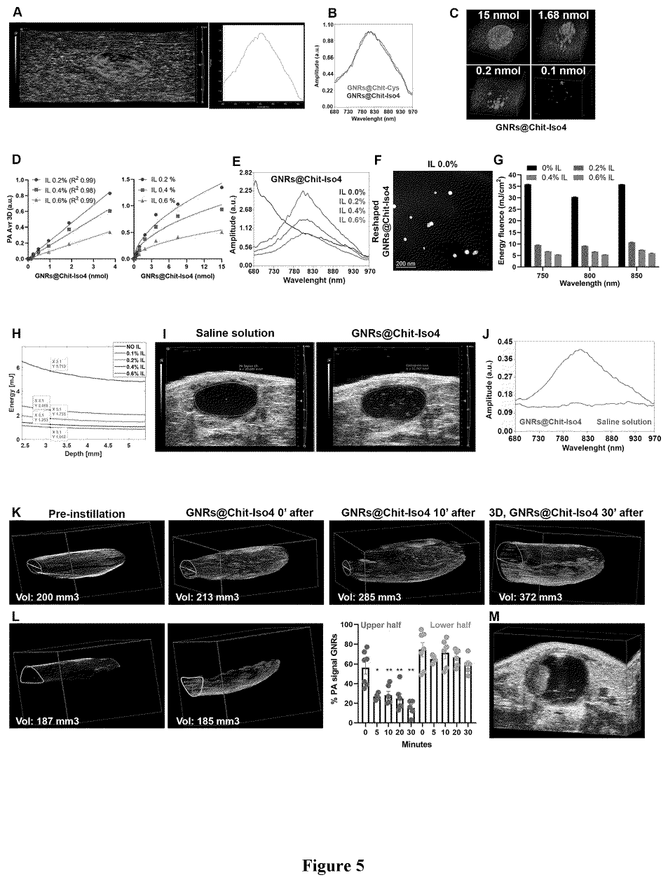

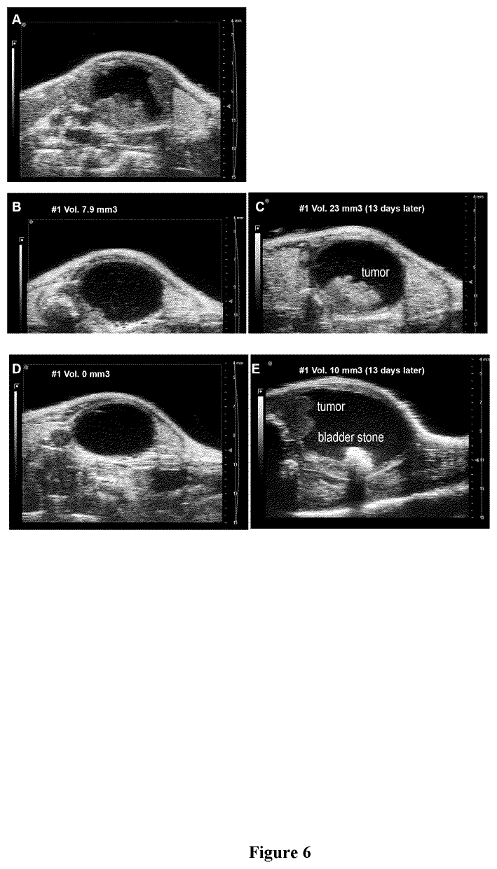

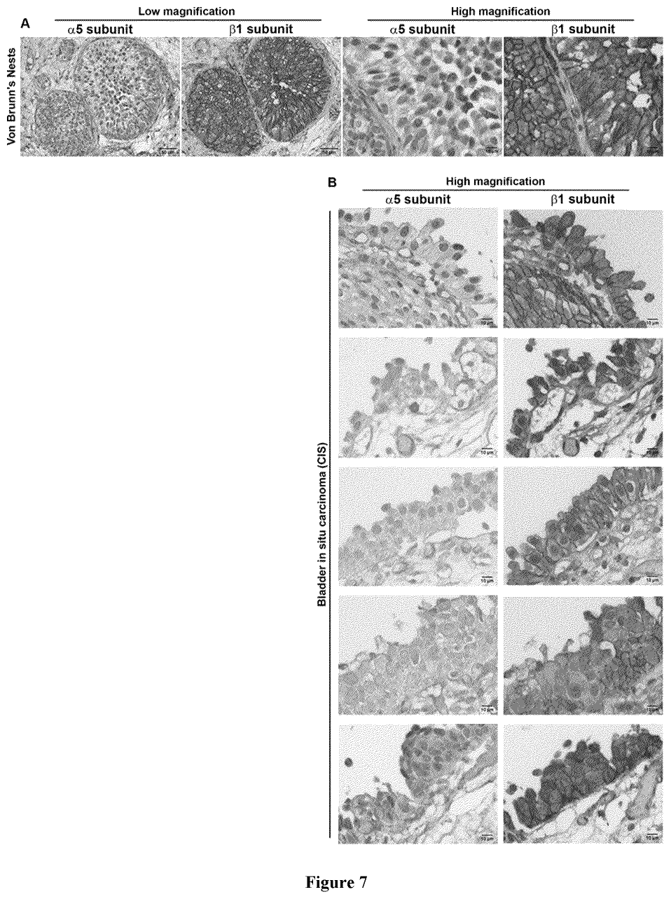

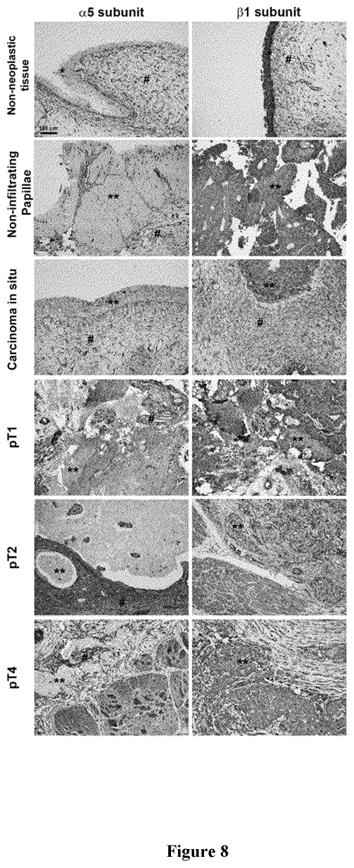

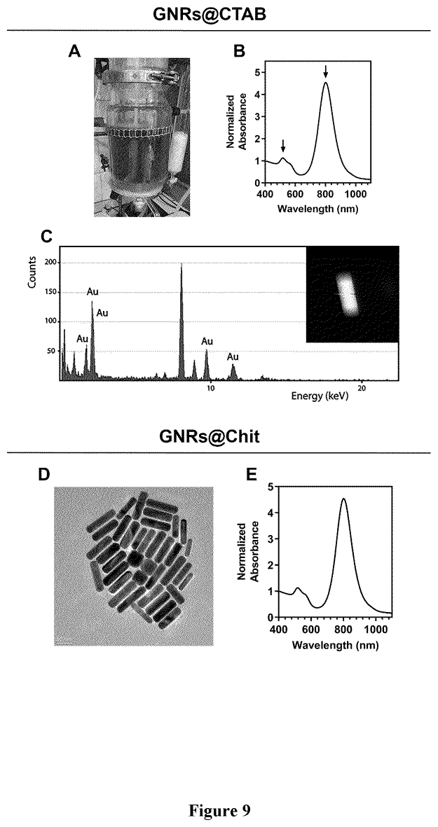

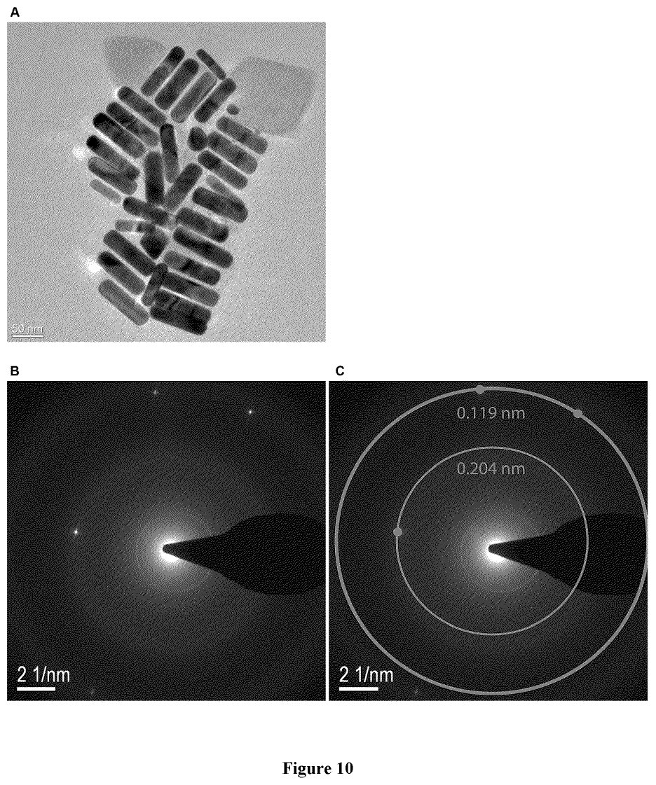

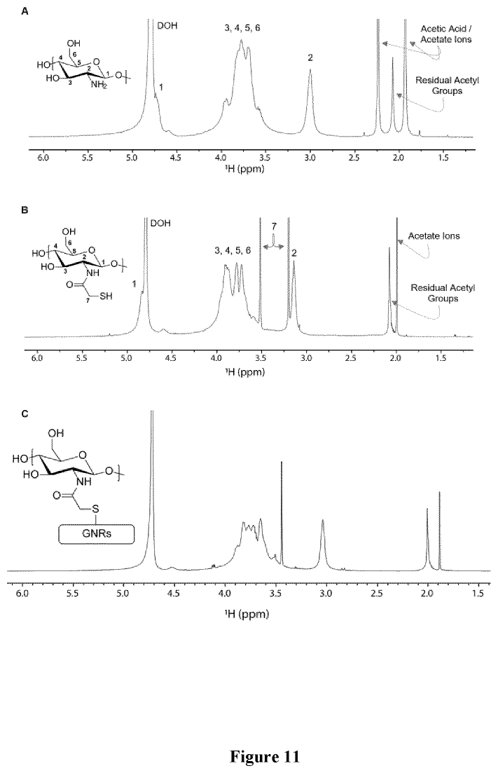

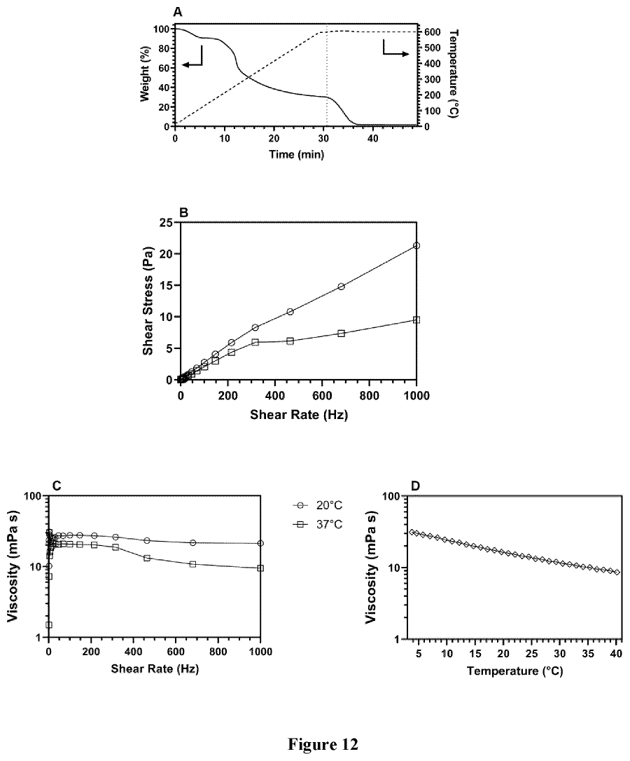

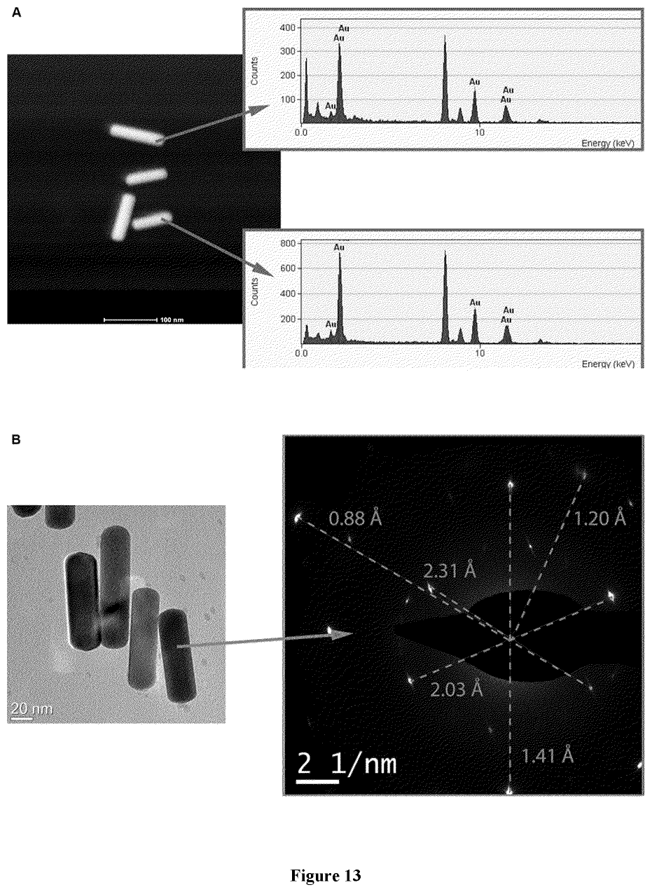

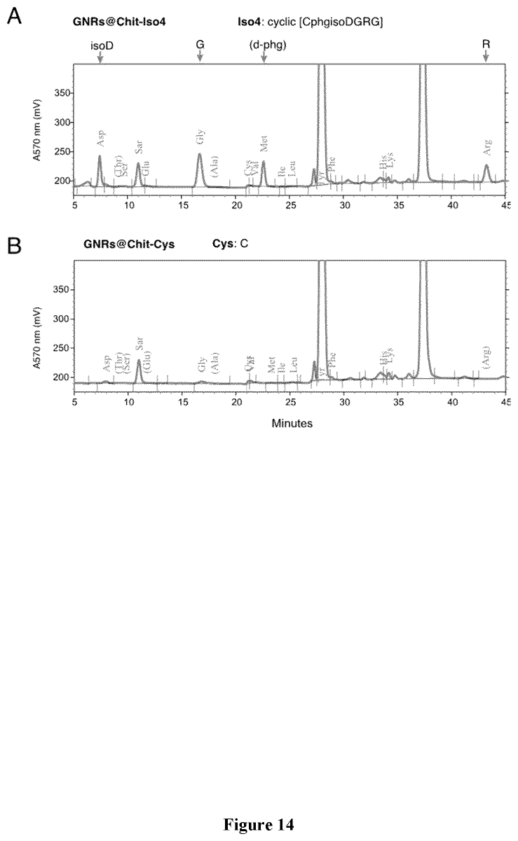

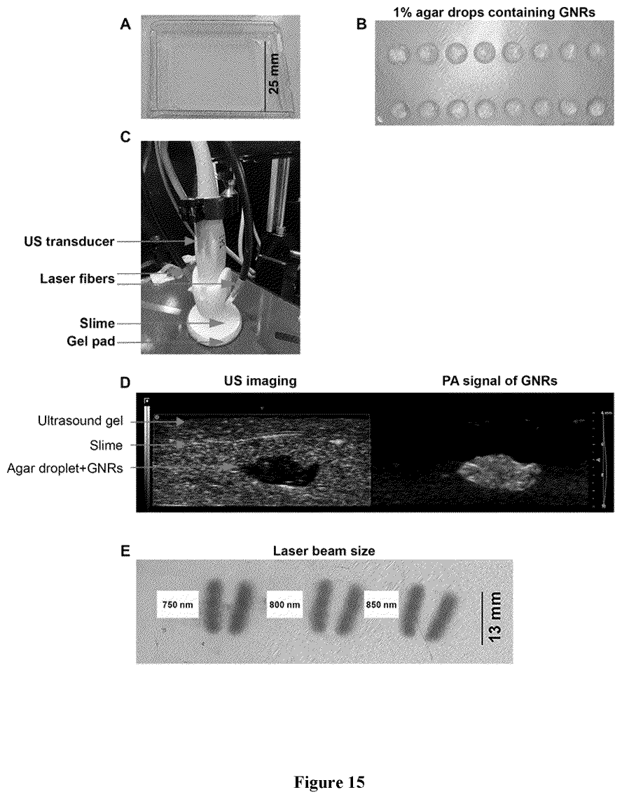

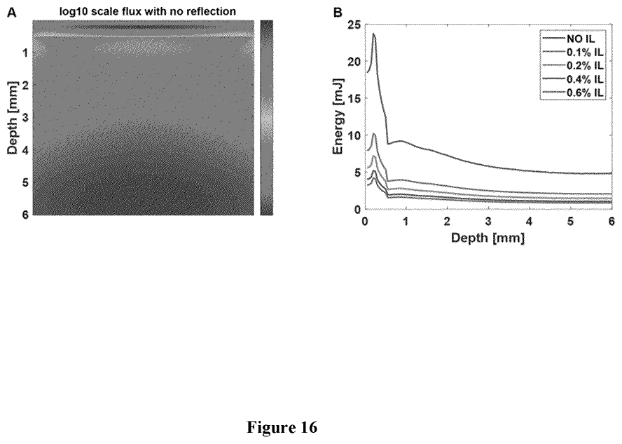

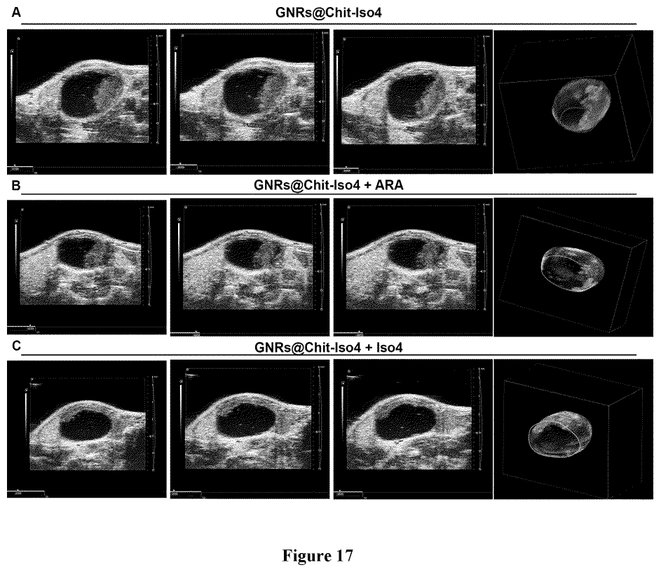

In order to do that the inventors coupled Iso4 of sequence [CphgisoDGRG] to fluorescent nanoparticles (Quantum Dots, Qdot) via the sulfhydryl group of the cysteine, and evaluated the binding of this conjugate (Iso4-Qdot) to MB49-Luc and 5637 cells. An irrelevant head-to-tail cyclized c(CGARAG) peptide was also coupled to Qdot (ARA-Qdot) and used as a negative control. Flow cytometry and fluorescence microscopy experiments showed that Iso4-Qdot, but not the ARA-Qdot, bound these cells with a potency that correlates with the expression level of α5β1 on these cells (FIGS. 2A and B).

Next, given that peptides containing isoDGR coupled to human serum albumin (HSA) can promote and support endothelial cell adhesion [25, 36], Iso4 was coupled to HSA and tested the conjugate (Iso4-HSA) in cell adhesion assays using MB49-Luc and 5637 cells. The results showed that Iso4-HSA, but not the activated HSA lacking the peptide (*HSA), promoted and supported cell adhesion (FIG. 2C), suggesting that both cell lines express functional Iso4 receptors, likely α5β1. Taken together, these results suggest that Iso4 can be coupled with different compounds in a functional manner and exploited to target α5β1-positive bladder cancer cells. To have GNRs on hand that can be exploited for the PAI of α5β1-positive tumors, the inventors functionalized Chitosan-coated GNRs (GNRs@Chit) that were prepared from CTAB-coated GNRs (GNRs@CTAB) having a longitudinal surface plasmon resonance peak centred at 800 nm (FIG. 9-12). GNRs@Chit was functionalized with cyclo-CphgisoDGRG (Iso4), a selective ligand of the α5β1 integrin. To this aim, inventors exploited a heterobifunctional crosslinker reagent, composed of an ethylene oxide spacer (PEG₁₂) bearing a N-hydroxysuccinimidyl (NHS) ester to its extremities and a maleimide functional group (NHS-PEG₁₂-maleimide). The NHS ester terminus reacts with the free amino groups on chitosan ensuring the binding of the linker to the chitosan, while the maleimide group is available to react in a further step with the sulfhydryl group of Iso4. Iso4 was then coupled with activated GNRs@Chit, and control GNRs with cysteine (Cys) in place of Iso4 were also prepared. The resulting product was called GNRs@Chit-Iso4 and GNRs@Chit-Cys (FIG. 3A). Next, the physical-chemical properties of the metal core of GNRs@Chit-Iso4 were assessed, determining the longitudinal-LSPR to peak at 802 nm (FIG. 3B), shape and dispersion (FIG. 3C), and aspect ratio of 3.62±0.67 (length 90.2±7.2 nm, width 24.9±2.6 nm, FIG. 3D). These findings show that conjugation of Iso4 does not modify the physical-chemical properties of GNRs, with only small and gradual variation of the longitudinal-LSPR peak vs GNRs@Chit (800 nm) and GNR@CTAB (798 nm) that is representative of the variation of the chemical environment surrounding the GNRs. Both the Cys- and the Iso4-conjugated nanosystems displayed a reduction in the Z-potential (+10.3 mV and +10.6 mV for GNRs@Chit-Iso4 and GNRs@Chit-Cys, respectively, vs. +40 mV measured for GNRs@Chit), according to the reduced amount of free amino groups on chitosan due to the presence of the PEG linker.

Moreover, the crystallinity and the composition of the GNRs@Chit-Iso4 was proven to be conserved after the consecutive conjugation steps, additionally revealing no detectable Br atoms from CTAB (FIG. 13) GNRs@Chit-Iso4 was therefore aliquoted in vials, freeze-dried and vacuum sealed to achieve 50 sterile single use vials containing the lyophilized product which results in a GNRs@Chit-Iso4 concentration of 1 mM when dissolved in 500 μL of water (FIG. 3E).

The stability of GNRs@Chit-Iso4 in human urine was then investigated at 37° C. for up to 2 h of incubation. No significant change in the shape of the LSPR spectrum occurred upon incubation in urine up to 2 h (FIG. 3F), with a 14% reduction of the absorbance at ˜800 nm observed in the first 40 minutes, and a further 7% reduction in the following 80 minutes of incubation (FIG. 3F). These results suggest that GNRs@Chit-Iso4 can be incubated at 37° C. in urine for at least 2 h without a dramatic loss of its optical properties in the NIR. The same techniques were employed for the characterization of GNRs@Chit-Cys, revealing no appreciable discrepancies with respect to GNRs@Chit-Iso4 (Results are reported in table 3).

TABLE 3

| | GNRs | | | |
|---|---|---|---|---|
| Type of analysis | GNRs@CTAB | GNRs@Chit | GNRs@Chit-Cys^{a} | GNRs@Chit-Iso4^{a} |
| VIS-NIR | | | | |
| λₘₐₓ (nm) ^{b} | 798 | 800 | 802 | 802 |
| TEM | | | | |
| GNRs length (nm) | — | 88.2 ± 6.4c | 90.2 ± 7.2d | 90.2 ± 7.2d |
| GNRs width (nm) | — | 25.7 ± 2.0c | 24.9 ± 2.6d | 24.9 ± 2.6d |
| Aspect Ratio ^{e} | — | 3.4 ± 0.5 | 3.6 ± 0.6 | 3.6 ± 0.6 |
| Zeta potential (mV) | +29.8 mV | +40 mV | +10.6 ± 0.4 | +10.3 ± 0.4 |
| FAAS ^{f} | | | | |
| Au (%) ^{g} | — | — | 2.01 | 1.97 |
| Au (mM) | — | — | 1.00 | 1.00 |
| GNRs/ml ^{h} | — | — | ~2 × 10¹¹ | ~2 × 10¹¹ |
| Gravimetric analysis | | | | |
| Dry matter (mg) ⁱ | — | — | 4.90 | 5.00 |

^{a}After resuspension of a lyophilized vial with 0.5 ml of water.
^{b} λₘₐₓ: wavelength of max absorbance.
cn = 150 by TEM.
dn = 300 by TEM.
^{e} Calculated as the length/width ratio.
^{f} After redispersion of the content of one vial in 0.5 mL of water.
^{g} Calculated as the ponderal ratio between gold content and total dry mass per vial.
^{h} Calculated approximating the GNRs as half-sphere-capped cylinders.
ⁱ Weight of the dry content of each vial.

To quantify the amount of peptide loaded onto GNRs@Chit-Iso4, the inventors measured the total amino acid composition present in the supernatant of GNRs@Chit-Iso4 after acidic hydrolysis. Hydrolysed GNRs@Chit-Cys was used as a negative control since it is expected to be an “amino acid-free” compound FIG. 14). The results, shown in table 4, of the quantification of GNRs@Chit-Iso4 revealed that ˜1×10¹¹ nanoparticles are loaded with about ˜0.5 mg peptide, which corresponds to ˜6×10⁶ peptides per GNR

TABLE 4

| | | Peptide | Peptide | Coupling | | Peptide density |
|---|---|---|---|---|---|---|
| | Batch | added | found | Efficiency | No of | (No of |
| Gold nanorods | code | (mg)^{b} | (mg) | (%) | peptides/GNR | peptides/μm²)^{d} |
| GNRs@Chit-Iso4 | #A | 4.74 | 4.3 ± 0.1 | 91 ± 2 | 8.1 (±0.4) × 10⁶ | 9.0 × 10⁸ |
| | | | (2) ^{c} | | | |
| GNRs@Chit-Iso4 | #B | 21 | 14.6 ± 0.5 | 69 ± 2 | 4.2 (±0.1) × 10⁶ | 4.7 × 10⁸ |
| | | | (2) ^{c} | | | |

a) Lyophilized GNRs@Chit-Iso4 were resuspended in water (0.5 mM of Au, 1 × 10¹¹ NPs/ml) at 5 mg/ml (based on their dry-matter content) and subjected to acidic hydrolysis (20 hours at 110° C., in 6M hydrochloric acid, 0.1% phenol, 0.1% thioglycolic acid under reduced pressure in an atmosphere nitrogen). The amino acid content in the hydrolyzed products was then quantified using ion exchange chromatography and post-column derivatization with ninhydrin. The hydrolyzed product obtained from GNRs@Chit-Cys was used to establish the background
^{b}By Ellman's quantification.
^{c} Number of independent quantifications.
^{d}The density of peptides per μm² of GNR was calculated considering each GNR as cylinders capped with two half-spheres and a surface of 0.009 μm². Data shown as mean ± SE.

Next, to demonstrate the presence of functional Iso4 on GNRs, the inventors measured the capability of GNRs@Chit-Iso4 and GNRs@Chit-Cys to promote cell-adhesion. To this aim, microtiter plates were coated with various amounts of GNRs@Chit-Iso4 or GNRs@Chit-Cys and seeded with MB49-Luc and 5637 cells. GNRs@Chit-Iso4, but not GNRs@Chit-Cys, induced cell adhesion and spreading of both cell lines (FIG. 4A), suggesting that the conjugation of Iso4 to GNRs@Chit does not affect the peptide function. Of note, the effective concentration 50 (EC₅₀) of GNRs@Chit-Iso4 for both cell lines was similar, being 0.73±0.05 μg/ml for MB49-Luc cells and 1.3±0.6 μg/ml for 5637 cells (FIG. 4A). The addition of an excess of free Iso4 peptide completely inhibited cell adhesion, suggesting that the adhesion was specific and dependent on the Iso4 peptide being coupled with GNRs (FIG. 4B).

The inventors then proceeded to investigate the effect of urine on the binding of GNRs@Chit-Iso4 to the α5β1 integrin and MB49-Luc cells. Intravesically administered GNRs@Chit-Iso4 is expected to target bladder tumor cells in a harsh environment characterized by the presence of urine and a broad variety of metabolites [37], bacteria [38], bacteria-derived mucus and floating urothelial cells that might impair the binding capability of GNRs@Chit-Iso4 to α5β1. With the intention of using targeted GNRs in the bladder with a protocol similar to that used for the photodynamic diagnosis performed using Hexvix [39], it was envisaged bladder draining before the intravesical instillation of GNRs, thus expecting that instilled GNRs would mix with increasing amounts of urine over time.

Thus, first it was investigated the impact of human urine on the binding of Iso4 to purified α5β1. To this aim, Iso4 was coupled with maleimide-activated horseradish peroxidase (HRP) to produce an Iso4-HRP conjugate to be used as a probe in direct binding assays with microtiter plates coated with α5β1. An HRP control conjugate using the ARA peptide instead of Iso4 was also prepared (ARA-HRP). As expected, Iso4-HRP, but not ARA-HRP, could bind α5β1 in a dose dependent manner (FIG. 4C), suggesting that the probe is functional. Of note, to recapitulate the urine environment we omitted the addition of any detergent in the binding and washing buffers. The effects of various amounts of human urine (range 11.5-90%) were then tested. The results showed that urine partially inhibited the binding of Iso4-HRP, with the overall recovery of binding being about 55% in 90% of urine (FIG. 4D), proving that unexpectedly Iso4 can still bind to α5β1 even in this unfavorable condition.

We further investigated the effects of human urine on the capability of GNRs@Chit-Iso4 to bind to MB49-Luc cells. To this aim, the effects of various amounts of urine on MB49-Luc cell adhesion to GNRs@Chit-Iso4-coated plates was tested. In parallel, GNRs@Chit-Cys-coated plates were used as negative controls to monitor the unspecific cell adhesions. Surprisingly, when MB49-Luc cells were seeded in DMEM medium containing 25-75% urine, a significant increase (˜20%) of cell adhesion to GNRs@Chit-Iso4, but not to GNRs@Chit-Cys, or to the control plate (lacking nanoparticles), was observed (FIG. 4E). The gain of cell adhesion to GNRs@Chit-Iso4 caused by the low concertation of urine was even more marked when the assays were carried in Hepes buffer supplemented with divalent ions (Mg²⁺/Mn²⁺) (FIG. 4F).

These results prove that unexpectedly urine does not prevent the capability of Iso4 grafted onto GNRs to bind to bladder cancer cells. Since GNRs are susceptible to shape change and a consequent loss of photoacoustic properties when stimulated with pulsed light above a certain energy threshold, light attenuators were used to reduce laser fluence to avoid the reshaping of the GNRs. The in vitro PA properties of the GNRs were investigated using agar drops containing GNRs@Chit-Iso4 or GNRs@Chit-Cys (15 nmol Au), and 0.6% Intralipid (IL) based light attenuators mounted on the tip of the optical fibers of the Vevo LAZR-X (FIG. 15). The results showed similar PA signals and spectrum for GNRs@Chit-Iso4 or GNRs@Chit-Cys, with maximum peaks at 810 nm (FIGS. 5A and 5B), and about 0.2 nmol of Au was detected using 0.6% IL (FIG. 5C). Next, it was assessed the minimum amount of IL required for the detection of the highest PA signal and the correct spectra of GNRs in vitro. Using light attenuators containing 0.2, 0.4 and 0.6% IL, it was found that the PA signal intensity of GNRs@Chit-Iso4 was inversely proportional to the amount of IL (FIG. 5D). As expected, GNRs@Chit-Iso4 analyzed using light attenuators without IL underwent reshaping, as observed by the change of shape of the PA spectrum (FIG. 5E) and further supported by the transition from the rod-like (FIG. 3) to a spherical shape (FIG. 5F).

In the absence of light attenuators, the energy fluence at the wavelength of 750, 800 and 850 nm (i.e., optical absorption of deoxy-blood, GNR@Chit-Iso4 and oxy-blood) was 30-35 mJ/cm². In the presence of 0.2% IL the energy fluence dropped to 9 mJ/cm² and further decreased with higher concentrations of IL (FIG. 5G). These findings indicate that the maximum PA signal from GNRs is obtained by irradiation with energy fluence of 9 mJ/cm².

In consideration of using the GNRs in vivo, because of the presence of tissues surrounding the murine bladder, it was considered to use light attenuators with lower amount of IL. Using a Monte Carlo simulation it was estimated the light energy at 800 nm wavelength that reaches the murine bladder, considering a distance of 0.3 cm from the skin to the bottom of the bladder.

It was considered the light energy that was measured for estimating the light fluence in FIG. 5G, and at a depth of 0.3 cm the energy value in the absence of light attenuators was estimated to be 5.3 mJ, while using the light attenuator made of 0.1% IL the energy value was 2.458 mJ (FIG. 5H, FIG. 16)

The inventors then proceeded to setup the conditions for the detection of the PA signal of GNR in the murine bladder. Light attenuators containing 0.1% IL were selected for imaging GNRs@Chit-Iso4 instilled in the murine bladder. A “dotted-like” PA signal was clearly visible in the murine bladder instilled with GNRs@Chit-Iso4 (10 nmol Au) but not with a 100 μl vehicle (saline solution) (FIG. 5I). The PA spectrum shape of these “dotted-like” structures was similar to that observed in vitro, i.e., with a peak of 810 nm (FIG. 5J). This result suggests that GNRs@Chit-Iso4 can be easily detected in vivo without any significant changes in the PA properties of the nanoparticles.

It was identified that 2.458 mJ represents the energy value that allows to obtain the maximum PA signal of GNRs in the murine bladder in the absence of reshaping. Time-course PAUS analysis of GNRs@Chit-Iso4 (10 nmol) at 0, 5, 10, 20 and 30 min after installation showed a progressive signal accumulation toward the bottom bladder (FIG. 5K). In particular, starting from 5 minutes after installation only ˜25% of PA signal could be detected in the upper half of the bladder, and most of the signal remained stacked in the lower half of the bladder (FIG. 5L), a condition that did not allow for the detection of a tumor that was located in the upper/lateral part of the bladder (FIG. 5M). Of note, no settlement of GNRs was observed upon storage at 37° C. up to 2 h, suggesting that GNRs maintain their colloidal properties outside of the bladder. The inventors with the intention of providing a technological platform for the early diagnosis of cancer located everywhere in the bladder tried to keep GNRs in suspensions in the bladder by means of manual handling of urine obtained through aspiration of urinary content with a syringe connected with the catheter and re-injection every 4 minutes for 3 times.

The particles can be successfully maintained in suspension and moved by different means, i.e. manual, physical, magnetic, ultrasound-assisted moving.

It was verified that manual handling of urine stable GNRs@Chit-Iso4 could be exploited for the diagnosis of the well-established orthotopic α5β1⁺ bladder tumor (i.e., the murine MB49-Luc derived tumor). To this aim, PAI was performed after the intravesical instillation of GNRs@Chit-Iso4 or GNRs@Chit-Cys, followed by three cycles of manual handling of the urinary content and two intravesical washes to remove the unbound GNRs. We observed that the binding of GNR@Chit-Iso4 was to the three well-established neoplastic lesions at the bottom, lateral side and upper part of the bladder, and not present in non-neoplastic tissue (FIG. 6A). Accordingly, no binding of GNR@Chit-Iso4 was observed in healthy mice or of GNR@Chit-Cys in tumor bearing mice suggesting that the Iso4 peptide was crucial for the efficient recognition of α5β1 tumor cells. Interestingly, the binding of GNR@Chit-Iso4 to MB49-Luc tumors was reduced by co-administration of an excess of free Iso4 peptide, but not ARA peptide (FIG. 19) confirming that the binding of GNR@Chit-Iso4 to α5β1 cells was mediated by the Iso4 peptide.

Next, it was verified whether our technological platform could be exploited to detected small superficial tumors. To this aim, it was analyzed the binding of GNR@Chit-Iso4 in a mouse bearing a small orthotopic tumor (i.e., 4 days after MB49-Luc cell implantation) Two representative animals are reported, showing that the GNR@Chit-Iso4 were able to detect i) a tumor with a volume of 7.9 mm³ (equal to 79 μl) that growth in the following days (FIG. 6B, 6C), and ii) a neoplastic region in the upper region of the bladder that was undetectable by US scan (FIG. 6D) but that give rise to tumor in the following days (FIG. 6E). These data suggest that the developed technological platform allows for the recognition of small and flat bladder cancers that are not detectable using bioluminescence or US imaging.

Finally the inventors demonstrate that nano constructs are reproducible and stable also successfully testing aliquots stored at +4° C. for >3 year.

The inventors therefore demonstrated the safety and feasibility of the platform to detect tumor lesions in the bladder. Designing nanoparticles and in particular gold nanorods linked with a peptide targeting α5β1 integrin they were able to correctly identify α5β1 integrin positive bladder tumors, such as the human bladder CIS.

The main clinical limitations of the approach for tumor imaging was related to the heterogeneity of tumor markers expressed among bladder cancer patients and the depth reached by the PAI. It was for example found that α5β1 expression in six out of eight (75%) specimens from patients with a diagnosis of bladder CIS, similar to what was previously reported for Cytokeratin 20 [52] in the bladder CIS or for EPCAM and PAR in MIBC [53]; the combination of 2 or more targeting ligands, either coupled to the same or different GNRs, might be exploited to reach all bladder CIS. In this preclinical study a transducer with center frequency 40 MHz was used, which facilitates a spatial resolution of 40 μm and an imaging depth of 15 mm; to move to the clinic, lower frequency transducers will be investigated to achieve the imaging depth required for the human studies. In an animal model of bladder cancer GNRs according to the invention were able to provide the early detection of bladder lesions <1 mm in size in patients. Due to the heat releasing properties of the GNRs, these particles also open novel avenues for the early detection and therapy of bladder cancer [33, 54].

The results of this study show the feasibility of the early diagnosis of bladder cancer using the α5β1-targeted GNRs@Chit-Iso4 conjugate as a photoacoustic contrast agent. Considering that the α5β1 integrin is also expressed by endometrial tumors [55], gastric cancer [56], breast cancer [57], and by the tumor neovasculature [58, 59], the photoacoustic imaging approach described here is exploitable in the diagnosis of other solid neoplasia.

The particles according to the invention are used in the photothermal ablation of solid tumors.

Cystoscope will instill GNRs and deliver light close to the urothelium. With ultrasound guidance an optical probe (on a Cystoscope) will be moved along the entire bladder to identify small lesions revealed by Photoacoustic imaging of the GNRs bound to the tumors. The delivery of the continuous light will irradiate the targeted area with the bound GNR. Assisted photothermal therapy is generated by the excitation of particles at a wavelength of 808 nm.

### Examples

Reagents: Bovine serum albumin (BSA) fraction-V, lipopolysaccharide from Escherichia coli 0111:B4, and all the other reagents, if not specified, were from Sigma Aldrich (St. Louis, MO). Medical grade, endotoxin-free, chitosan prepared from Snow Crab (deacetylation degree 87.6% and viscosity 66 mPa·s at 20° C.) was from ChitoLytic (St. John's, Newfoundland, Canada). Maleimide-PEG₁₂-NHS (1-maleimide-3-oxo-7,10,13,16,19,22,25,28,31,34,37,40-dodecaoxa-4-azatritetracontan-43-oic acid succinimidyl ester, 99%) was from Iris Biotech GmbH (Marktredwitz, Germany). Wild-type human integrin α5β1 (octyl β-D-glucopyranoside preparation) was from Immunological Sciences (Rome, Italy). Head-to-tail cyclized peptides c(CphgisoDGRG) and c(CGARAG), called Iso4 and ARA, respectively, were from Biomatik (Delaware, USA). The identity and purity of Iso4 and ARA were confirmed through mass spectrometry and HPLC analyses (expected/found monoisotopic mass, MH⁺, Da/Da, were 622.35/622.40 and 516.23/561.23, respectively; purity >95% in both cases). Peptides were dissolved in sterile water and stored in aliquots at −80° C. Human serum albumin (HSA) was from Baxter (Deerfield, IL). The Iso4-HSA conjugate (Iso4 chemically coupled to HSA via 4-(N-maleimidomethyl)cyclohexane-1-carboxylic acid 3-sulfo-N-hydroxysuccinimide ester sodium salt (sulfo-SMCC)), and *HSA (HSA activated with sulfo-SMCC and quenched with β-mercaptoethanol instead of Iso4) were prepared as described [26].

Synthesis of GNRs@CTAB with maximal absorption at 800 nm. Cetyltrimethylammonium bromide (CTAB)-coated GNRs (GNRs@CTAB) with a maximum absorption of 800 nm, were prepared according to a previously published procedure with minor modifications [1] and scaled to a volume of ˜2 litres. The initial solution for the growth of GNRs was prepared by dissolving CTAB (31.14 g, 85.4 mmol) and sodium oleate (4.28 g, 14.0 mmol) in 1.8 L of warm water (˜50° C.) in a thermostatically controlled 2-L jacketed reactor, equipped with mechanical stirring. When the solution reached 30° C., 810 μL of AgNO₃ solution (0.4 M in ultrapure water) were added and the mixture that was then incubated for 15 min without stirring. Then, under continuous stirring (700 rpm), 8.652 mL of HAuCl₄ solution (0.1 M in ultrapure water) were added. Au(III) was reduced to Au(I) using sodium oleate for 90 min, after which 3.56 mL of hydrochloric acid (37%) and 3.6 mL of ascorbic acid (0.079 M) were added to adjust the pH to 1.0 and to ensure the complete reduction of the gold precursor. Separately, a seed solution was prepared by dissolving 364 mg (1.0 mmol) of CTAB in 10 mL of warm water in a 50 mL round-bottomed flask. After cooling down to room temperature 25 μL of HAuCl₄ solution (0.1 M) were added while stirring. The seeds were formed by quickly injecting 600 μL of ice-cold sodium borohydride (0.01 M) into the solution, causing the colour of the solution to change from yellow to brown indicating the formation of ultra-small gold seeds. Finally, after ageing the seed solution for 30 minutes at room temperature, 690 μL of it were added to the growth solution, which was vigorously stirred for 30 s then left undisturbed overnight at 30° C. to allow for GNR growth. Purification of GNRs@CTAB was performed by i) centrifugation in 50-mL Falcon tubes (6,000 rpm for 100 min), ii) removal of 40 mL of the supernatant and iii) re-dispersion in 40 mL of ultrapure water, and the process was repeated 3 times. The final product was then collected in 200 ml of ultrapure water. The gold concentration in GNRs@CTAB was 2.09 mM, as determined by atomic absorption spectroscopy.

Coating of GNRs with chitosan (GNRs@Chit). Medical grade chitosan (500 mg, 3.1 mmol) was dissolved in 50 mL of 1 vol. % acetic acid and mixed with 0.5 mL (7.2 mmol) of thioglycolic acid under moderate stirring. N-(3-dimethylaminopropyl)-N′-ethylcarbodiimide hydrochloride (500 mg, 2.6 mmol) was then added to activate the carboxylic group of thioglycolic acid and to promote the coupling to the amino groups of chitosan. The reaction was left to incubate for 6 h at room temperature under continuous stirring. The product was then dialyzed using a 3.5 kDa cut-off dialysis tube overnight against ultrapure water and the resulting thiolated-chitosan was diluted to 500 mL with water. At this point, 30 mL of GNRs@CTAB were added dropwise under mild stirring and the resulting solution was incubated (48 h, room temperature) to allow for the coupling of thiolated-chitosan and GNRs. The product was subsequently concentrated using an Amicon® stirred cell equipped with PES membranes (100 kDa cut-off, using 4 bar nitrogen pressure) to remove CTAB, and the final product (60 mL), called GNRs@Chit, was then stored at +4° C. until the subsequent step.

Functionalization of GNRs@Chit with Iso4 or Cys: GNRs@Chit was prepared from cetyltrimethylammonium bromide (CTAB)-coated GNRs (GNRs@CTAB; synthesis and characterization detailed in the Supplementary Information). To functionalize GNRs@Chit with Iso4, 30 mL of Maleimide-PEG₁₂-NHS (1 mg/mL in ultrapure water, mol=29.7 mg) were mixed with 30 mL of GNR@Chit (1 mM of Au, 30 mol=5.909 mg Au) under stirring to achieve a final weight ratio of Maleimide-PEG₁₂-NHS:Au=5:1. The mixture was then left to incubate overnight at room temperature and then dialyzed against ultrapure water using a 3.5 kDa cut-off dialysis tube for 24 h, at room temperature, to remove the excess crosslinker. The product (65 mL), consisting of activated GNRs@Chit-PEG₁₂-maleimide, was then mixed with the Iso4 peptide (21 mg, 34 mol, 10 mg/mL in ultrapure water) and left to react for 24 h at room temperature. The unreacted maleimide groups were then quenched by adding an excess of cysteine hydrochloride (18 mg, 102 mol). The product, called GNRs@Chit-Iso4, was then dialyzed against ultrapure water, the Au content quantified and the product aliquoted in vials containing 50 g of Au was freeze-dried and stored at −80° C. In parallel, control nanoparticles with cysteine instead of Iso4 were prepared as described above, but using an excess of cysteine (36 mg, 204 mol) instead of the Iso4 peptide. This product was called GNRs@Chit-Cys. About 50 vials of both products were prepared.

Characterization of the physicochemical properties of GNRs@CTAB, GNRs@Chit and functionalized GNRs@Chit-Iso4: Gold concentration was determined by flame atomic absorption spectroscopy (FAAS) using a SpectraAA 100 Varian spectrometer (Agilent Technologies, Santa Clara, USA). Gold nanorods (100 μL) were dissolved in aqua regia (3 mL) and diluted to 10 mL with ultrapure water prior to analysis. For the calibration of the FAAS analysis, Au standard solutions at 1, 2, 5 and 10 mg/L were prepared by diluting the appropriate amounts of 1000 mg/mL TraceCERT® solutions in 30% aqua regia.

VIS-NIR absorption spectra (range 400-1100 nm) were recorded using a Cary 3500 UV-VIS-NIR modular spectrometer (Agilent Technologies, Santa Clara, USA) using a 1 cm path-length plastic cuvette.

Transmission Electron Microscopy (TEM) was performed using a TEM/STEM FEI TECNAI F20 operating at 200 keV equipped with a probe for energy-dispersing x-ray spectroscopy (EDX), selected-area electron diffraction (SAED) and High Angle Annular Dark Field Detector (HAADF). Before the analysis, samples were placed on a continuous-carbon film, supported on a copper grid and dried at 120° C.

¹H-NMR spectra were obtained using a Varian Inova NMR spectrometer (14.09 T, 600 MHz). The chemical shifts were reported in ppm of frequency relative to the residual solvent signals (1H-NMR: 4.80 ppm for heavy water).

Viscosity measurements were performed using an MCR102 (Anton-Parr, Graz, Austria) modular compact rheometer with a DPP25-SNO geometry, i.e. a double plate geometry with a diameter of 25 mm.

Zeta potential measurements were performed using a Zetasizer-nano-S (Malvern Panalytical, Malvern, UK) in DTS1060C-Clear disposable zeta cells, at 25° C.

Thermogravimetric analysis (TGA) was performed using a Q600 thermoscale (TA Instruments, New Castle, USA) working in nitrogen atmosphere from room temperature to 600° C. with a heating ramp of 20° C./min, then switched to air and kept at 600° C. for 15 min.

Stability of GNRs@Chit and GNRs@Chit-Iso4 in human urine: The stability of GNRs@Chit-Iso4 in human urine was assessed by diluting the GNR solution (0.725 mM of Au in 10 ml of water) with human urine samples (10 ml) collected from 10 healthy adult volunteers (4 males and 6 females) and placed under stirring in a water bath at 37° C. At various time points, 1 mL of the mixture was withdrawn, diluted in 10 mL of cold water (+4° C.) and subjected to VIS-NIR. A urine solution (50% in water) was used as the blank reference in the spectrometric analysis.

Cell lines: Two human primary bladder epithelial cells (ATCC catalog number PCS-420-010; CELLnTEC catalog number HBLAK) were cultured according to the manufacturer's instructions and used at passage four. Human bladder cancer cell lines RT4, 5637, and HT-1376 were from ATCC (catalog number HTB-2™, HTB-9™, HTB-4™, CRL-1472™, respectively), RT112 cell line was from Merck (catalog number 85061106). These cell lines were cultured in RPMI medium (Gibco; Thermo Fisher Scientific) with standard supplements. Murine bioluminescent MB49-Luc cells were kindly provided by Prof. Carla Molthoff (VU University Medical Center, The Netherlands) and cultured in DMEM medium (Gibco; Thermo Fisher Scientific) with standard supplements. For this cell line, a cell bank was prepared and authenticated for lack of cross-contamination by analyzing 9 short tandem repeats DNA [27] (IDEXX Bioanalytics, Ludwigsburg, Germany). A vial of cell bank was used to start a new experiment. The cells were routinely tested for Mycoplasma contamination and cultured for not more than 4 weeks before use.

Human data collection: Human data collection followed the principles outlined in the Declaration of Helsinki. Patients signed an informed consent agreeing to supply their own anonymous information and tissue specimens for future studies. The study was approved by the Institutional Review Board (Ethic Committee IRCCS Ospedale San Raffaele, Milan, Italy). All methods were carried out in accordance with the approved guidelines.

Surgical specimens were staged according TNM classification [28]. Paired non-tumoral and tumoral bladder areas were from the same bladder of an individual submitted to TURB or radical cystectomy for bladder cancer; six TURBs with a histological diagnosis of CIS and two bladders with non-muscle invasive bladder cancer (NMIBC; CIS, pTa, pT1) and muscle invasive bladder cancer (MIBC; pT2-pT4) were used.

Immunohistochemical analysis: Tissue samples, fixed in 10% buffered formalin for 24-48 h at room temperature and then embedded in paraffin, were stained as previously reported [29]. Briefly, 3 μm tissue sections were dehydrated according to standard procedures and boiled twice in 0.25 mM EDTA pH 8.0 for 5 min using a microwave oven (780 W). After washing with PBS, and quenching the endogenous peroxidase with 3% H₂O₂, tissue sections were incubated with anti-α5 or -β1 rabbit monoclonal antibodies (see table 5 below) for 2 h at room temperature.

TABLE 5

| | | Antibody | | | Antibody | Vendor |
|---|---|---|---|---|---|---|
| Antigen | Host | clone | Reactivity ^{b)} | Dilution | Isotype | (catalog) |
| α5 | Rabbit | EPR7854 ^{a)} | h/m/r | 1/100 | IgG | Abcam |
| | | | | | | (ab15031) |
| β1 | Rabbit | EPR16895 ^{a)} | h/m/r | 1/1000 | IgG | Abcam |
| | | | | | | (ab179471) |
| None | Rabbit | Polyclonal | None | — | IgGs ^{c)} | Primm |

^{a)} Monoclonal antibody.
^{b)} h/m/r, human, mouse and rat (according to technical data sheet).
^{c)} Isotype-matched negative control antibodies were prepared through Protein-A Sepharose purification.

After washing, the binding of rabbit primary antibodies was detected using the Universal HRP-Polymer Biotin-free detection system (MACH4, BioCare Medical, USA) and 3,3-diaminobenzidine free base (DAB) as a chromogen. Tissue samples were then counterstained with Harris' hematoxylin.

FACS analysis: The staining of α5 and β1 integrins expressed on the cell surface was carried out as described [30] using 5 μg/ml of the monoclonal antibodies listed in Table 6. Isotype-matched antibodies were used as negative controls. The binding of primary antibodies was detected using Alexa Fluor 488-labeled goat anti-mouse or -hamster secondary antibodies according to their animal species.

TABLE 6

| | | Antibody | | Antibody | Vendor |
|---|---|---|---|---|---|
| Antigen | Host | (clone) | Reactivity ^{b)} | Isotype | (catalog) |
| α5 | Mouse | P1D6 | h | IgG₃ | Merk |
| | | | | | (MAB-1956z) |
| β1 | Mouse | P5D2 | h | IgG₁ | Merck |
| | | | | | (MAB1959z) |
| None ^{a)} | Mouse | MOPC-21 | None | IgG | Sigma |
| | | | | | (M 5284) |
| α5 | Hamster | HMα5-1 | m, r | IgG | Biolegend |
| | | | | | (103902) |
| β1 | Hamster | HMβ1-1 | m, h | IgG | Biolegend |
| | | | | | (142602) |
| None ^{a)} | Hamster | None | None | IgG | eBioscience |
| | | | | | (14-4888-85) |

^{a)} Isotype-matched negative control antibody.
^{b)} h/m/r, human, mouse and rat (according to Technical Data Sheet).

Binding assays of Iso4-Qdot to MB49-Luc and 5637 cell lines: Iso4 and ARA were coupled to amino-modified quantum dots nanoparticles, Qdot605 ITK Amino PEG (Thermo Fischer) as previously described [30]. The binding of Iso4-Qdot and ARA-Qdot to MB49-Luc and 5637 cells was assessed through FACS and fluorescence microscopy experiments. FACS analysis was carried out as follows: the cells were detached with DPBS containing 5 mM EDTA pH 8.0 (DPBS-E), washed with DPBS, and suspended in 25 mM Hepes buffer, pH 7.4, containing 150 mM sodium chloride, 1 mM magnesium chloride, 1 mM manganese chloride, 1% BSA (binding buffer-1) and Iso4- or ARA-Qdot (range 30-0 nM, 5×10⁵ cells/100 μl tube). After 2 h of incubation at 37° C., the cells were washed with binding buffer-1 without BSA and fixed with 4% formaldehyde. Bound fluorescence was detected using a CytoFLEX S cytofluorimeter (Beckman Coulter). The binding of Iso4-Qdot and ARA-Qdot to living 5637 cells was analyzed as follows: 5637 cells were cultured in a 96-well clear bottom black plate (5×10⁴ cells/well) for 48 h, 5% CO₂, 37° C. The plates were washed with binding buffer-1 and incubated with Iso4-Qdot or ARA-Qdot solution (30 nM in binding buffer-1) for 2 h at 37° C., 5% CO₂. The cells were then washed with binding buffer-1, and fixed with 3% paraformaldehyde and 2% sucrose for 20 min. Bound fluorescence was acquired using the Cellomics ArrayScan XTI Studio Scan (Thermo Fischer Scientific) system.

α5β1 integrin binding assay: Iso4 and ARA were chemically conjugated to maleimide-activated HRP (InnovaBioscience, cat. 401-0002), via thiol group, as follows: a vial of the lyophilized product was suspended in 1 ml of PBS (10 mM sodium phosphate buffer, pH 7.4, 138 mM sodium chloride, 2.7 mM potassium chloride, Sigma, P-3813) containing 5 mM EDTA (PBS-E); the solution was then divided into 2 aliquots and mixed with Iso4 or ARA peptide, using a 3:1 peptide: enzyme ratio (mol/mol). The final products were called Iso4-HRP and ARA-HRP. The conjugates were diluted (range 0-400 nM) in 25 mM Tris-HCl, pH 7.4, containing 150 mM sodium chloride, 1 mM magnesium chloride, 1 mM manganese chloride and 1% BSA (binding buffer-2) and added to 96-well plate polyvinyl chloride (PVC) microtiter plates (Carlo Erba, cod. FA5280100) coated with or without α5β1 (4 μg/ml) and left to incubate for 2 h. The plates were washed with 25 mM Tris-HCl, pH 7.4, containing 150 mM of sodium chloride, 1 mM of magnesium chloride, 1 mM of manganese chloride, and bound peroxidase was detected by adding a chromogenic solution (o-phenylenediamine dihydrochloride, OPD). The chromogenic reaction was stopped by adding 1 N sulfuric acid. The absorbance at 490 nm was then measured using a microtiter plate reader. The effect of human urine (from a healthy donor) on the binding of Iso4-HRP was studied as described above by mixing Iso4-HRP (300 nM final concentration) and various amounts of urine diluted in binding buffer-2.

Cell adhesion assays: 96-well PVC microtiter plates were coated with Iso4-HSA or GNRs@Chit-Iso4 in 50 mM sodium phosphate, pH 7.3, containing 150 mM sodium chloride, (overnight incubation at 4 C). The plates were washed and blocked with 2% BSA in DMEM or RPMI-1640 (200 μl/well, 1 h). MB49-Luc and 5637 cells were detached with DPBS-E, washed twice with DPBS, and suspended in DMEM or RPMI-1640 containing 0.1% BSA (binding buffer-3), and added to the coated-plates (1.5×10⁵ cells/well, 100 μl). After 1-2 h of incubation at 37° C., 5% CO2, the plates were washed with binding buffer-3. Adherent cells were fixed and stained with 0.5% crystal violet in 20% methanol. After washing with water, the dye was extracted from cells using a 10% acetic acid solution (140 μl/well) and the absorbance at 570 nm was measured using a microplate reader. The effect of urine on the pro-adhesive properties of GNRs@Chit-Iso4 was investigated as described above, except that the cells were seeded in binding buffer-3 or binding buffer-1 and various amounts of human urine (obtained from a healthy volunteer).

Quantification of Iso4 peptide bound to GNRs@Chit: The amount of Iso4 loaded on GNRs@Chit-Iso4 was quantified by measuring the total aminoacidic contents after acidic hydrolysis of the nanoparticles by Alphalyse Inc., Denmark.

Murine orthotopic bladder tumor model: All procedures and studies involving mice were approved by the Institutional Animal Care and Use Committee of San Raffaele Scientific Institute and performed according to the prescribed guidelines (IACUC, approval number 942). Female albino C57BL/6J mice (9 weeks old, weighing about 20 g, Charles River Laboratories, Italy) were anesthetized with ketamine (80 mg/kg) and xylazine (15 mg/kg) and kept in dorsal position. Using a 24-gauge catheter, the bladder of each mouse was emptied and instilled with or without MB49-Luc cells (10⁵ cells/100 μl in DPBS). Thirty minutes later the catheter was removed, and mice were allowed to recover and returned to their cage. Tumor growth was monitored by measuring the tumor volume through US imaging (see below) and in vivo bioluminescent quantification after administration of luciferin (15 mg/kg, intra peritoneum) using the non-invasive In Vivo Imaging System (IVIS, PerkinElmer, USA).

US and PAI of GNRs@Chit and GNRs@Chit-Iso4: High-resolution US and PA imaging have been acquired using the Vevo LAZR-X platform (FUJIFILM VisualSonics, Inc., Toronto, ON, Canada). The imaging platform includes a high frequency US system (Vevo 3100) combined with an Nd:YAG nano-second pulsed laser with repetition rate of 20 Hz. The linear US transducer array Mx 550D consists of 256 elements with a nominal center frequency of 40 MHz (25-55 MHz bandwidth) and a spatial resolution of 40 μm with a maximum imaging depth of 15 mm. Light from the laser is delivered to the tissue through optical fibers mounted on either side of the transducer. During volumetric US-PA acquisitions, a stepper motor is used for the linear translation of the US transducer and optical fibers along the sample. The linear stepper motor moves in steps of a minimum of 0:1 mm while capturing 2-D parallel images, for a maximum 3D range distance of 6.4 cm.

3D B mode scan was carried out for in vitro (drop) and in vivo (mouse bladder) studies. The photoacoustic spectra between 680 nm and 970 nm were scanned with a step size of 5 nm; for the in vivo studies (murine bladder) the 3D multispectral PA scans were acquired selecting PA spectral curve of tissue components melanin, deoxy- and oxy-generated blood, and GNRs; the processed wavelengths (680; 722; 764; 810; 924; 970 nm) were automatically selected from the spectral curve used to spectrally unmix the GNRs signal from other endogenous tissue chromophore signals such as oxy, deoxy hemoglobin. The algorithm reported by Luke et al [31] was used to select these wavelengths which is ideal for separating the signal from GNRs from other endogenous absorbers. For the in vitro studies (agar drops embedded in slime) the 3D multispectral PA scans were acquired selecting the PA spectral curve of slime and GNRs (processed wavelengths 680; 782; 810 nm).

Data analyses were conducted using the Vevo® Lab software; volumes of interest were obtained by manually drawing Volumes of Interest (VOIs) on 3D B-mode images. GNRs, melanin, oxy- and deoxy-hemoglobin content were estimated through spectral unmixing analyses of the spectroscopic data.

Light attenuators. Since the GNRs are susceptible to change in shape at the higher laser threshold, light attenuators were prepared to reduce the laser fluence to avoid GNRs reshaping. Light attenuators were prepared as follows: agar powder (cat. A9539, Sigma) was suspended in deionized and distilled water (1% final concentration), melted at 95° C., and mixed with different concentration of Intralipid (cat. 1141, Sigma). The mixtures (3 ml) were poured into Disposable Base Molds (30×24×5 mm, Bio-Optica, Milan, Italy) left to solidify for 2 min at room temperature, and stored in a humidified chamber until used. The light attenuator was then sliced and mounted in contact with the optical fibers.

In vitro PAI of GNRs was carried out as follows: GNRs (30 μl in DPBS with calcium and magnesium) were mixed with a 1% agar solution (30 μl), the mixture was then poured on Parafilm® M (Sigma) and left to solidify in a humidified chamber (FIG. 15). The solidified product (called “agar drop”) was then placed on an ultrasound gel pad (Aquaflex, Parker), embedded in slime made of polyvinyl alcohol polymer crosslinked with sodium tetraborate (Barrel of slime) (FIG. 15) and PAI was performed using the two optical fiber bearing light attenuators. The transducer was perpendicularly placed on the object under investigation previously covered with ultrasound transmission gel (Aquasonic 100, Parker). Axial sections were acquired using the following settings for B-Mode: 2D Power 100%; 2D Gain 13 dB; for PA-mode: Pa Power 100%; PA gain 44 dB; TGC and depth were maintained identical for all drops. PA and US data have been analyzed using VevoLab 3.2.5 software (FIG. 15).

Light fluence. The energy of the laser at the wavelength of 750, 800 and 850 nm was measured for 2 minutes using a laser energy meter (PE50BF-DIFH-C, P/N 7Z02943, Ophir, Germany). The laser beam size was assessed by shooting the laser for 5 seconds onto a piece of photographic paper (Kodak Linagraph Type 1895) placed 8 mm from the light source. The resulting burned area was then quantified with a ruler (FIG. 15) and the light fluence was calculated by dividing the light energy by the light beam size.

Simulation of the energy transport in the murine bladder. A numerical simulation of the light fluence was performed to estimate the energy distribution within the tissue domain. The light energy distribution was obtained by implementing the Monte Carlo model of light transport, based on the MCXLAB computer simulation. The optical properties (the absorption coefficient (μₐ), the scattering coefficient (μₛ), the scattering anisotropic factor (g), and the refractive index (n)) of the skin, tissue surrounding the bladder, urine and GNRs used for the simulation were recently described [33], and reported in table 7. Fluence simulation was carried out with 109 photons at 800 nm, considering a Gaussian light source within a 120×120 pixel domain of tissue.

TABLE 7

| | Skin | Standard | | |
|---|---|---|---|---|
| | line¹ | tissue¹ | GNRs¹ | Urine¹ |
| Absorption coefficient | 0.4275 | 0.1 | 42.93 | 0.002 |
| (μa) [1/mm] | | | | |
| Scattering coefficient (μs) | 25 | 10 | 0.521 | 1 |
| Scattering anisotropic factor (g) | 0.9 | 0.9 | 0.9 | 1 |
| Refractive index (n) | 1.33 | 1.44 | 1.36 | 1.333 |

For the urine, the absorption coefficient, scattering coefficient, and refractive index were assumed to be the same as those of water; the scattering anisotropic factor was chosen to be 1, implying that photons propagate inside the urine but do not undergo scattering. Values for the skin, standard tissue and GNRs from recent publication [5]

Ethics approval and consent to participate: Human data collection followed the principles outlined in the Declaration of Helsinki. Patients signed an informed consent agreeing to supply their own anonymous information and tissue specimens for future studies. The study was approved by the Institutional Review Board (Ethic Committee IRCCS Ospedale San Raffaele, Milan, Italy). All methods were carried out in accordance with the approved guidelines. All procedures and studies involving mice were approved by the Institutional Animal Care and Use Committees of San Raffaele Scientific Institute and performed according to the prescribed guidelines (IACUC, approval number 942).

### REFERENCES

[1] TNM classification of malignant tumors. UICC International Union Against Cancer. 8th edn., G. M. Brierley J D, Wittekind C., Editor. Wiley-Blackwell and UICC: New York, USA. (2017).
[2]M. Burger, J. W. Catto, G. Dalbagni, H. B. Grossman, H. Herr, P. Karakiewicz, W. Kassouf, L. A. Kiemeney, C. La Vecchia, S. Shariat, Y. Lotan, Epidemiology and risk factors of urothelial bladder cancer, Eur Urol 63(2) (2013) 234-41.
[3] M. Babjuk, M. Burger, E. M. Comperat, P. Gontero, A. H. Mostafid, J. Palou, B. W. G. van Rhijn, M. Roupret, S. F. Shariat, R. Sylvester, R. Zigeuner, O. Capoun, D. Cohen, J. L. D. Escrig, V. Hernandez, B. Peyronnet, T. Seisen, V. Soukup, European Association of Urology Guidelines on Non-muscle-invasive Bladder Cancer (TaT1 and Carcinoma In Situ)—2019 Update, Eur Urol (2019).
[4] B. W. van Rhijn, M. Burger, Y. Lotan, E. Solsona, C. G. Stief, R. J. Sylvester, J. A. Witjes, A. R. Zlotta, Recurrence and progression of disease in non-muscle-invasive bladder cancer: from epidemiology to treatment strategy, Eur Urol 56(3) (2009) 430-42.
[5] E. Ozden, A. T. Turgut, K. Turkolmez, B. Resorlu, M. Safak, Effect of bladder carcinoma location on detection rates by ultrasonography and computed tomography, Urology 69(5) (2007) 889-92.
[6] https://uroweb.org/guideline/non-muscle-invasive-bladder-cancer/#5, (2020).
[7] P. Zapala, B. Dybowski, S. Poletajew, L. Bialek, A. Niewczas, P. Radziszewski, Clinical rationale and safety of restaging transurethral resection in indication-stratified patients with high-risk non-muscle-invasive bladder cancer, World J Surg Oncol 16(1) (2018) 6.
[8] C. Yeung, T. Dinh, J. Lee, The health economics of bladder cancer: an updated review of the published literature, PharmacoEconomics 32(11) (2014) 1093-104.
[9] W. W. Liu, P. C. Li, Photoacoustic imaging of cells in a three-dimensional microenvironment, J Biomed Sci 27(1) (2020) 3.
[10] I. Steinberg, D. M. Huland, O. Vermesh, H. E. Frostig, W. S. Tummers, S. S. Gambhir, Photoacoustic clinical imaging, Photoacoustics 14 (2019) 77-98.
[11] M. L. James, S. S. Gambhir, A molecular imaging primer: modalities, imaging agents, and applications, Physiol Rev 92(2) (2012) 897-965.
[12] Z. Hosseinaee, M. Le, K. Bell, P. H. Reza, Towards non-contact photoacoustic imaging [review], Photoacoustics 20 (2020) 100207.
[13] Y. Zhou, G. Li, L. Zhu, C. Li, L. A. Cornelius, L. V. Wang, Handheld photoacoustic probe to detect both melanoma depth and volume at high speed in vivo, J Biophotonics 8(11-12) (2015) 961-967.
[14]A. K. Yadav, S. Hernandez, S. Su, J. Chan, Acoustic-based chemical tools for profiling the tumor microenvironment, Curr Opin Chem Biol 57 (2020) 114-121.
[15]P. Wang, T. Ma, M. N. Slipchenko, S. Liang, J. Hui, K. K. Shung, S. Roy, M. Sturek, Q. Zhou, Z. Chen, J. X. Cheng, High-speed intravascular photoacoustic imaging of lipid-laden atherosclerotic plaque enabled by a 2-kHz barium nitrite raman laser, Sci Rep 4 (2014) 6889.
[16]Z. Xu, Q. Zhu, L. V. Wang, In vivo photoacoustic tomography of mouse cerebral edema induced by cold injury, J Biomed Opt 16(6) (2011) 066020.
[17]J. Weber, P. C. Beard, S. E. Bohndiek, Contrast agents for molecular photoacoustic imaging, Nat Methods 13(8) (2016) 639-50.
[18]M. Maturi, E. Locatelli, I. Monaco, M. Comes Franchini, Current concepts in nanostructured contrast media development for in vivo photoacoustic imaging, Biomater Sci 7(5) (2019) 1746-1775.
[19]H. Hamidi, J. Ivaska, Every step of the way: integrins in cancer progression and metastasis, Nat Rev Cancer 18(9) (2018) 533-548.
[20]M. Nieberler, U. Reuning, F. Reichart, J. Notni, H. J. Wester, M. Schwaiger, M. Weinmuller, A. Rader, K. Steiger, H. Kessler, Exploring the Role of RGD-Recognizing Integrins in Cancer, Cancers (Basel) 9(9) (2017).
[21]L. M. Knowles, J. Zewe, G. Malik, A. V. Parwani, J. R. Gingrich, J. Pilch, CLT1 targets bladder cancer through integrin alpha5beta1 and CLIC3, Mol Cancer Res 11(2) (2013) 194-203.
[22]T. Saito, M. Kimura, T. Kawasaki, S. Sato, Y. Tomita, Correlation between integrin alpha 5 expression and the malignant phenotype of transitional cell carcinoma, Br J Cancer 73(3) (1996) 327-31.
[23] https://www.proteinatlas.org/ENSG00000115414-FN1/pathology/urothelial+cancer.
[24]A. Brunner, A. Tzankov, The role of structural extracellular matrix proteins in urothelial bladder cancer (review), Biomark Insights 2 (2007) 418-27.
[25]F. Curnis, A. Sacchi, R. Longhi, B. Colombo, A. Gasparri, A. Corti, IsoDGR-Tagged Albumin: A New ?v?3 Selective Carrier for Nanodrug Delivery to Tumors, Small 9(5) (2013) 673-678.
[26]F. Curnis, A. Sacchi, R. Longhi, B. Colombo, A. Gasparri, A. Corti, IsoDGR-tagged albumin: a new alphavbeta3 selective carrier for nanodrug delivery to tumors, Small 9(5) (2013) 673-8.
[27]W. K. Yung, The value of cell line validation, Neuro-oncology 14(6) (2012) 675.
[28]M. J. Magers, A. Lopez-Beltran, R. Montironi, S. R. Williamson, H. Z. Kaimakliotis, L. Cheng, Staging of bladder cancer, Histopathology 74(1) (2019) 112-134.
[29]L. Genovese, L. Zawada, A. Tosoni, A. Ferri, P. Zerbi, R. Allevi, M. Nebuloni, M. Alfano, Cellular localization, invasion, and turnover are differently influenced by healthy and tumor-derived extracellular matrix, Tissue Eng Part A 20(13-14) (2014) 2005-18.
[30]F. Nardelli, M. Ghitti, G. Quilici, A. Gori, Q. Luo, A. Berardi, A. Sacchi, M. Monieri, G. Bergamaschi, W. Bermel, F. Chen, A. Corti, F. Curnis, G. Musco, A stapled chromogranin A-derived peptide is a potent dual ligand for integrins alphavbeta6 and alphavbeta8, Chem. Commun. (Camb.) 55(98) (2019) 14777-14780.
[31]G. P. Luke, S. Y. Nam, S. Y. Emelianov, Optical wavelength selection for improved spectroscopic photoacoustic imaging, Photoacoustics 1(2) (2013) 36-42.
[32]W. Thielicke, E. J. Stamhuis, PIVlab—Towards User-friendly, Affordable and Accurate Digital Particle Image Velocimetry in MATLAB, Journal of open research software 2(1):e30 (2014).
[33]E. H. Ooi, V. Popov, M. Alfano, J. K. K. Cheong, Influence of natural convection on gold nanorods-assisted photothermal treatment of bladder cancer in mice, Int J Hyperthermia 37(1) (2020) 634-650.
[34]T. C. M. Zuiverloon, F. C. de Jong, J. C. Costello, D. Theodorescu, Systematic Review: Characteristics and Preclinical Uses of Bladder Cancer Cell Lines, Bladder Cancer 4(2) (2018) 169-183.
[35]C. Scheepbouwer, S. Meyer, M. J. Burggraaf, J. Jose, C. F. Molthoff, A Multimodal Imaging Approach for Longitudinal Evaluation of Bladder Tumor Development in an Orthotopic Murine Model, PLoS One 11(8) (2016) e0161284.
[36]F. Curnis, A. Sacchi, A. Gasparri, R. Longhi, A. Bachi, C. Doglioni, C. Bordignon, C. Traversari, G.-P. Rizzardi, A. Corti, Isoaspartate-glycine-arginine: A new tumor vasculature-targeting motif, Cancer Res. 68(17) (2008) 7073-7082.
[37]S. Bouatra, F. Aziat, R. Mandal, A. C. Guo, M. R. Wilson, C. Knox, T. C. Bjorndahl, R. Krishnamurthy, F. Saleem, P. Liu, Z. T. Dame, J. Poelzer, J. Huynh, F. S. Yallou, N. Psychogios, E. Dong, R. Bogumil, C. Roehring, D. S. Wishart, The human urine metabolome, PLoS One 8(9) (2013) e73076.
[38]F. Pederzoli, R. Ferrarese, V. Amato, I. Locatelli, E. Alchera, R. Luciano, M. Nebuloni, A. Briganti, A. Gallina, R. Colombo, A. Necchi, M. Clementi, F. Montorsi, N. Mancini, A. Salonia, M. Alfano, Sex-specific Alterations in the Urinary and Tissue Microbiome in Therapy-naive Urothelial Bladder Cancer Patients, Eur Urol Oncol 3(6) (2020) 784-788.
[39] https://uroweb.org/guideline/non-muscle-invasive-bladder-cancer/#note_228.
[40]L. A. Sordillo, Y. Pu, S. Pratavieira, Y. Budansky, R. R. Alfano, Deep optical imaging of tissue using the second and third near-infrared spectral windows, J Biomed Opt 19(5) (2014) 056004.
[41]K. H. Song, L. V. Wang, Deep reflection-mode photoacoustic imaging of biological tissue, J Biomed Opt 12(6) (2007) 060503.
[42]S. K. Balasubramanian, J. Jittiwat, J. Manikandan, C. N. Ong, L. E. Yu, W. Y. Ong, Biodistribution of gold nanoparticles and gene expression changes in the liver and spleen after intravenous administration in rats, Biomaterials 31(8) (2010) 2034-42.
[43]K. E. Ibrahim, M. G. Al-Mutary, A. O. Bakhiet, H. A. Khan, Histopathology of the Liver, Kidney, and Spleen of Mice Exposed to Gold Nanoparticles, Molecules 23(8) (2018).
[44]F. Pederzoli, M. Bandini, D. Raggi, L. Marandino, G. Basile, M. Alfano, R. Colombo, A. Salonia, A. Briganti, A. Gallina, F. Montorsi, A. Necchi, Is There a Detrimental Effect of Antibiotic Therapy in Patients with Muscle-invasive Bladder Cancer Treated with Neoadjuvant Pembrolizumab?, Eur Urol 80(3) (2021) 319-322.
[45]Y. Zhao, F. Ye, T. B. Brismar, X. Li, R. He, R. Heuchel, R. El-Sayed, N. Feliu, W. Zheng, S. Oerther, J. Dutta, W. J. Parak, M. Muhammed, M. Hassan, Multimodal Imaging of Pancreatic Ductal Adenocarcinoma Using Multifunctional Nanoparticles as Contrast Agents, ACS Appl Mater Interfaces (2020).
[46]B. Seo, K. Lim, S. S. Kim, K. T. Oh, E. S. Lee, H. G. Choi, B. S. Shin, Y. S. Youn, Small gold nanorods-loaded hybrid albumin nanoparticles with high photothermal efficacy for tumor ablation, Colloids Surf B Biointerfaces 179 (2019) 340-351.
[47]B. Shan, H. Wang, L. Li, G. Zhou, Y. Wen, M. Chen, M. Li, Rationally designed dual-plasmonic gold nanorod@cuprous selenide hybrid heterostructures by regioselective overgrowth for in vivo photothermal tumor ablation in the second near-infrared biowindow, Theranostics 10(25) (2020) 11656-11672.
[48]A. Aziz, J. Holthof, S. Meyer, O. G. Schmidt, M. Medina-Sanchez, Dual Ultrasound and Photoacoustic Tracking of Magnetically Driven Micromotors: From In Vitro to In Vivo, Adv Healthc Mater (2021) e2101077.
[49]A. Giannantoni, S. M. Di Stasi, M. B. Chancellor, E. Costantini, M. Porena, New frontiers in intravesical therapies and drug delivery, Eur Urol 50(6) (2006) 1183-93; discussion 1193.
[50]M. Racioppi, L. Di Gianfrancesco, M. Ragonese, G. Palermo, E. Sacco, P. F. Bassi, ElectroMotive drug administration (EMDA) of Mitomycin C as first-line salvage therapy in high risk “BCG failure” non muscle invasive bladder cancer: 3 years follow-up outcomes, BMC Cancer 18(1) (2018) 1224.
[51]B. A. John, N. Said, Insights from animal models of bladder cancer: recent advances, challenges, and opportunities, Oncotarget 8(34) (2017) 57766-57781.
[52]K. A. Lombardo, B. Murati Amador, V. Parimi, J. Hoffman-Censits, W. Choi, N. M. Hahn, M. Kates, T. J. Bivalacqua, D. McConkey, M. O. Hoque, A. Matoso, Urothelial Carcinoma In Situ of the Bladder: Correlation of CK20 Expression With Adaptive Immune Resistance, Response to BCG Therapy, and Clinical Outcome, Appl Immunohistochem Mol Morphol (2020).
[53]V. M. Baart, G. van der Horst, M. M. Deken, S. S. Bhairosingh, T. Schomann, V. Q. Sier, M. H. van der Mark, L. Iamele, H. de Jonge, M. Resnati, A. P. Mazar, R. C. M. Pelger, G. van der Pluijm, P. J. K. Kuppen, A. L. Vahrmeijer, C. F. M. Sier, A multimodal molecular imaging approach targeting urokinase plasminogen activator receptor for the diagnosis, resection and surveillance of urothelial cell carcinoma, Eur J Cancer 146 (2021) 11-20.
[54]J. K. Cheong, V. Popov, E. Alchera, I. Locatelli, M. Alfano, L. Menichetti, P. Armanetti, M. Maturi, M. C. Franchini, E. H. Ooi, Y. S. Chiew, A numerical study to investigate the effects of tumour position on the treatment of bladder cancer in mice using gold nanorods assisted photothermal ablation, Comput Biol Med 138 (2021) 104881.
[55]Y. Xu, Y. Li, J. Pan, X. Kang, X. Zhang, X. Feng, S. Li, C. Li, J. Zhang, C. Li, G. Wang, EM2D9, a monoclonal antibody against Integrin a5b1, has potent antitumor activity on endometrial cancer in vitro and in vivo, Cancer Lett (2020).
[56]J. Ren, S. Xu, D. Guo, J. Zhang, S. Liu, Increased expression of alpha5beta1-integrin is a prognostic marker for patients with gastric cancer, Clin Transl Oncol 16(7) (2014) 668-74.
[57]H. Yao, D. M. Veine, D. L. Livant, Therapeutic inhibition of breast cancer bone metastasis progression and lung colonization: breaking the vicious cycle by targeting alpha5beta1 integrin, Breast Cancer Res Treat 157(3) (2016) 489-501.
[58]F. Schaffner, A. M. Ray, M. Dontenwill, Integrin alpha5beta1, the Fibronectin Receptor, as a Pertinent Therapeutic Target in Solid Tumors, Cancers (Basel) 5(1) (2013) 27-47.
[59]L. A. Rocha, D. A. Learmonth, R. A. Sousa, A. J. Salgado, alphavbeta3 and alpha5beta1 integrin-specific ligands: From tumor angiogenesis inhibitors to vascularization promoters in regenerative medicine?, Biotechnol Adv 36(1) (2018) 208-227.

1. Metal based nanoparticles
coated with a polymer functionalized with thiol groups and —NH groups, said polymer preferably being selected among thiolated chitosan, thiolated and aminated alginic acid, thiolated and aminated hyaluronic acid, or a protein preferably selected from the group consisting of albumin and gelatin, or a synthetic thiolated and aminated polymer, preferably α-thio-ω-amino polyethylene glycols
wherein said groups are linked to a ligand of the integrin family receptors, preferably a peptide containing an integrin binding motif, and antibody or part of an antibody, a peptidomimetic or an aptamer, via a heterobifunctional crosslinker,
the crosslinker bearing functional groups able to bind to amino groups, preferably selected from the group consisting of N-hydroxysuccinimidyl ester group (NHS ester), an isocyanate group (—NCO), an isothiocyanate group (—NCS), a Sulfo-N-hydroxysuccinimidyl ester group (sulfo-NHS ester), or a carboxylic acid group which is connected by activation with carbodiimide coupling agents;
and/or a functional group able to bind thiol groups, preferably selected from the group consisting of: a maleimide group, a terminal vinyl group or a terminal alkyne group; and/or functional groups able to bind alkynes such as azides; and/or functional groups able to bind azides, such as alkynes.

2. The metal based nanoparticles according to claim 1 wherein the metal is selected from the group consisting of gold, silver, or hybrid gold/silver, and the nanoparticles have photoacoustic properties.

3. The metal based nanoparticles according to claim 1 wherein the nanoparticles are selected from the group consisting of nanosphere, nanorod, nanostar, nanocage, nanoprism or nanoshell or nanowires, nanoplates, and hallow shells.

4. The metal based nanoparticles according to claim 1 wherein the functionalized polymer is thiolated chitosan.

5. The metal based nanoparticles according to claim 1 wherein the ligand of the integrin family receptors is a peptide comprising the RGD or isoDGR motif.

6. The metal based nanoparticles according to claim 1 wherein the ligand of the integrin family receptors is a peptide selected from the group consisting of:
[XGisoDGRG], of SEQ ID No: 1
[XisoDGRGG], of SEQ ID No: 2
[XphgisoDGRG], of SEQ ID No: 3
[XGisoDGRphg], of SEQ ID No: 4
[XisoDGRphgG], of SEQ ID No: 5
[XisoDGRGphg] of SEQ ID No: 6
[CphgisoDGRG] peptide of SEQ ID NO: 7
XFETLRGDERILSILRHQNLLKELQD, of SEQ ID No: 8
XFETLRGDLRILSILRHQNLLKEL, of SEQ ID No: 9
and
XFETLRGDLRILSILRX₁QNLX₂KELQD, of SEQ ID No: 10
wherein “X” is selected from cysteine, lysine or a non-natural amino acid comprising an alkyne or azide group; and
X₁ and X₂ are respectively propargyl glycine and azidolysine joined via a triazole bridge.

7. The metal based nanoparticles according to claim 1 wherein the ligand of the integrin family receptors is the cyclic head-to-tail [CphgisoDGRG] peptide of SEQ ID NO: 7.

8. The metal based nanoparticles according to claim 1 wherein the crosslinker is maleimido-PEG₁₂-NHS ester.

9. The metal based nanoparticles according to claim 1 wherein
the nanoparticles are gold nanorods
the functionalized polymer is thiolated chitosan
the peptide is [CphgisoDGRG] peptide of SEQ ID No:7 and
the crosslinker is maleimido-PEG₁₂-NHS ester.

10. A composition comprising the metal based nanoparticles according to claim 1 and at least one of following solvent: water; physiologic solution; Dulbecco's Modified Eagle Medium (DMEM); Dulbecco's phosphate-buffered saline (DPBS), HEPES buffer, TRIS buffer, and PIPES buffer, each containing metal divalent ions.

11. The composition according to claim 10 comprising one or more antitumoral agents.

12. A kit comprising single use vials containing the metal based nanoparticles according to claim 1, a solvent or a buffer for resuspending the nanoparticles, optionally syringes and instruction for use.

13. A method of treatment of diagnosis of a patient comprising administering the metal based nanoparticles according to claim 1 to a patient in need of diagnosis and/or treatment in vivo.

14. The method according to claim 13 wherein the in vivo diagnosis and/or treatment is for solid tumors.

15. The method according to claim 14 wherein the solid tumor is selected among urothelial, bladder, gastroesophageal, colorectal, pancreatic, ovarian, lung, cervix, breast and renal cancer, brain tumors and hepatocellular carcinoma.

16. The method according to claim 13, wherein the treatment is photothermal therapy of solid tumors.

17. A method for ultrasound and photoacoustic imaging ex vivo which comprises the following steps:
a) applying the metal based nanoparticles according to claim 1 to the target tissue to be imaged
b) photoacoustic visualization of the target tissue, and
c) evaluating the visualized target tissue.

18. The method of claim 16, wherein the solid tumor is bladder cancer.


SEQUENCE LISTING (10 sequences)

SEQ ID NO: 1 - protein, 6 aa, synthetic construct
  VARIANT 1: note = cysteine, a lysine or any alkyne- or -azide functionalized amino acid such as propargylglycine or azidolysine
  SITE 3: note = L-isoaspartato
  UNSURE 1..6: note = head-to-tail cyclized
XGDGRG

SEQ ID NO: 2 - protein, 6 aa, synthetic construct
  VARIANT 1: note = cysteine, a lysine or any alkyne- or -azide functionalized amino acid such as propargylglycine or azidolysine
  SITE 2: note = L-isoaspartate
  UNSURE 1..6: note = head-to-tail cyclized
XDGRGG

SEQ ID NO: 3 - protein, 6 aa, synthetic construct
  VARIANT 1: note = cysteine, a lysine or any alkyne- or -azide functionalized amino acid such as propargylglycine or azidolysine
  SITE 2: note = d-phenylglycine
  SITE 3: note = L-isoaspartate
  UNSURE 1..6: note = head-to-tail cyclized
XGDGRG

SEQ ID NO: 4 - protein, 6 aa, synthetic construct
  VARIANT 1: note = a cysteine, a lysine or any alkyne- or -azide functionalized amino acid such as propargylglycine or azidolysine
  SITE 6: note = d-phenylglycine
  SITE 3: note = L-isoaspartate
  UNSURE 1..6: note = head-to-tail cyclized
XGDGRG

SEQ ID NO: 5 - protein, 6 aa, synthetic construct
  VARIANT 1: note = a cysteine, a lysine or any alkyne- or -azide functionalized amino acid such as propargylglycine or azidolysine
  SITE 2: note = L-isoaspartate
  SITE 5: note = d-phenylglycine
  UNSURE 1..6: note = head-to-tail cyclized
XDGRGG

SEQ ID NO: 6 - protein, 6 aa, synthetic construct
  VARIANT 1: note = a cysteine, a lysine or any alkyne- or -azide functionalized amino acid such as propargylglycine or azidolysine
  SITE 2: note = L-isoaspartate
  SITE 6: note = d-phenylglycine
  UNSURE 1..6: note = head-to-tail cyclized
XDGRGG

SEQ ID NO: 7 - protein, 6 aa, synthetic construct
  SITE 2: note = d-phenylglycine
  SITE 3: note = L-isoAspartate
  UNSURE 1..6: note = head-to-tail cyclized
CGDGRG

SEQ ID NO: 8 - protein, 26 aa, synthetic construct
  VARIANT 1: note = cysteine, a lysine or any alkyne- or -azide functionalized amino acid such as propargylglycine or azidolysine
XFETLRGDERILSILRHQNLLKELQD

SEQ ID NO: 9 - protein, 24 aa, synthetic construct
  VARIANT 1: note = a cysteine, a lysine or any alkyne- or -azide functionalized amino acid such as propargylglycine or azidolysine,
XFETLRGDLRILSILRHQNLLKEL

SEQ ID NO: 10 - protein, 26 aa, synthetic construct
  VARIANT 1: note = a cysteine, a lysine or any alkyne- or -azide functionalized amino acid such as propargylglycine or azidolysine,
  VARIANT 17: note = propargylglycine
  VARIANT 21: note = azidolysine
  SITE 17: note = residue 17 forms a triazole bridge with residue 21
XFETLRGDLRILSILRXQNLXKELQD